(19) United States
(12) Patent Application Publication
(10) Pub. No.: US 2026/0002207 A1
(43) Pub. Date: Jan. 1, 2026

(54) NUCLEIC ACID SEQUENCING METHODS WITH CONTROL SEQUENCES

(71) Applicant: 10x Genomics, Inc., Pleasanton, CA (US)

(72) Inventor: Justin COSTA, Union City, CA (US)

(21) Appl. No.: 19/249,939

(22) Filed: Jun. 25, 2025

Related U.S. Application Data
(60) Provisional application No. 63/665,884, filed on Jun. 28, 2024.

(51) Int. Cl. C12Q 1/6874; C12Q 1/6825; C12Q 1/6841

(52) U.S. Cl. CPC C12Q 1/6874; C12Q 1/6825; C12Q 1/6841

(57) ABSTRACT

The present disclosure relates in some aspects to methods, systems, and kits for nucleic acid sequencing using a control sequencing primer to perform a plurality of cycles of a nucleic acid sequencing reaction of a control sequence comprising a known sequence. In some cases, the control sequencing primer is detected to identify the location of a plurality of copies of the control sequence. In certain embodiments, for a cycle of the plurality of cycles at the identified location, the method comprises comparing a signal from the nucleic acid sequencing reaction to an expected signal from the known sequence, thereby determining noise in the signal and/or identifying an error in the nucleic acid sequencing reaction.

### CROSS-REFERENCE TO RELATED APPLICATIONS

This patent application claims priority benefit of U.S. Provisional Patent Application No. 63/665,884, filed Jun. 28, 2024, entitled “NUCLEIC ACID SEQUENCING METHODS WITH CONTROL SEQUENCES,” which is herein incorporated by reference in its entirety for all purposes.

### REFERENCE TO AN ELECTRONIC SEQUENCE LISTING

The content of the electronic sequence listing (202412021600seglist.xml; Size: 9,921 bytes; and Date of Creation: Jun. 23, 2025) is herein incorporated by reference in its entirety.

### FIELD

The present disclosure relates in some aspects to methods for sequencing nucleic acid molecules, including error correction for nucleic acid sequencing.

### BACKGROUND

Nucleic acid sequencing is a versatile tool that helps scientists advance the understanding of biology and has wide-ranging applications in various fields, such as medical diagnostics, biotechnology, forensic biology, and virology. Despite advances in nucleic acid sequencing, many challenges remain unaddressed, including accumulation of noise during nucleic acid sequencing reactions resulting in sequencing errors or failure to accurately call bases. The present application addresses these and other needs.

### SUMMARY

Of paramount importance for nucleic acid sequencing is the ability to detect the correct bound or incorporated nucleotide in a cycle of nucleic acid sequencing with high fidelity, followed by complete cleavage of a reversible terminator group to continue a sequencing reaction. In the case of sequencing-by-synthesis (SBS), complete cleavage of the fluorophore from a labeled nucleotide is also important to avoid accumulation of noise in an SBS reaction. Virtually any incompleteness of cleavage—either at the level of the fluorophore or the reversible terminator—will lead to phasing of the optical signatures detected in the sequencing reaction. The ability to monitor nucleic acid sequencing reactions (e.g., the efficiency of each incorporation step and completion of cleavage between sequencing cycles) would be of value to assay and algorithmic development, as well as informing the user performing the nucleic acid sequencing reaction of the quality of the nucleic acid sequencing results that will be obtained. In some embodiments, the provided methods allow for monitoring levels of noise in a nucleic acid sequencing reaction. In some embodiments, the provided methods comprise adjusting one or more thresholds in a base-calling algorithm used for the nucleic acid sequencing reaction

In some embodiments, provided herein is a method comprising: a) contacting a biological sample, wherein the biological sample comprises at a first location a plurality of copies of a control sequence comprising a known sequence, with sequencing primers comprising: (i) a control sequencing primer labeled with a first fluorescent dye, wherein the control sequencing primer binds adjacent to the control sequence, and (ii) a second sequencing primer, wherein the second sequencing primer binds adjacent to a sequence of interest; b) detecting the first fluorescent dye at the first location, thereby identifying the first location; c) performing a plurality of cycles of a nucleic acid sequencing reaction by extending the sequencing primers; and d) for a cycle of the plurality of cycles at the identified first location, comparing a signal from the nucleic acid sequencing reaction to an expected signal from the known sequence, thereby determining noise in the signal and/or identifying an error in the nucleic acid sequencing reaction.

In some embodiments, the plurality of cycles comprises at least four cycles. In some embodiments, at least one position of the known sequence is adenine (A), at least one position of the known sequence is thymine (T) or uracil (U), at least one position of the known sequence is guanine (G), and at least one position of the known sequence is cytosine (C). In some embodiments, at least one position of the known sequence is adenine (A), at least one position of the known sequence is thymine (T), at least one position of the known sequence is guanine (G), and at least one position of the known sequence is cytosine (C).

In some embodiments, the plurality of copies of the control sequence comprises locally amplified copies of the control sequence. In some embodiments, the plurality of copies of the control sequence are in a rolling circle amplification product. In some embodiments, the biological sample further comprises a plurality of copies of the sequence of interest. In some embodiments, the plurality of copies of the sequence of interest comprises locally amplified copies of the sequence of interest. In some embodiments, the plurality of copies of the sequence of interest are in a rolling circle amplification product.

In some embodiments, each cycle of the nucleic acid sequencing reaction comprises determining a fluorescent signal intensity indicative of incorporation or binding of a nucleotide of a particular nucleobase type at one or more locations comprising the first location. In some embodiments, the nucleic acid sequencing reaction is a sequencing-by-synthesis reaction. In some embodiments, c) (performing a plurality of cycles of a nucleic acid sequencing reaction by extending the sequencing primers) is performed before b) (detecting the first fluorescent dye at the first location, thereby identifying the first location). In some embodiments, b) is performed prior to, simultaneous with, or subsequent to the plurality of cycles of the nucleic acid sequencing reaction in c). In some embodiments, b) is performed in a plurality of cycles prior to, simultaneous with, or subsequent to the plurality of cycles of the nucleic acid sequencing reaction in c). In some embodiments, b) is performed before c), and the method comprises cleaving the first fluorescent dye from the control sequencing primer or quenching the first fluorescent dye before c).

In some embodiments, the first fluorescent dye is detectable in a first channel, and each cycle of the nucleic acid sequencing reaction comprises detecting a fluorescent signal intensity indicative of incorporation or binding of a nucleotide of a particular nucleobase type in additional channels comprising a second channel and a third channel.

In some embodiments, the set of nucleotides comprises first nucleotides having a first nucleobase type and a first label, a second nucleotides having a second nucleobase type and a second label, a mixture of third nucleotides having a third nucleobase type wherein 40-60% of the mixture has the first label and the remainder of the mixture has the second label, and a fourth nucleotide having a fourth nucleobase type and an absence of a label, and the first label is detectable in the second channel and a second label is detectable in the third channel. In some embodiments, the first and second labels are not detectable or are minimally detectable in the first channel. In some embodiments, the first fluorescent dye is detectable in a first channel, and the signal from the nucleic acid sequencing reaction comprises detecting a fluorescent signal intensity indicative of incorporation or binding of nucleotide of a particular nucleobase type in additional channels comprising a second channel, a third channel, and a fourth channel.

In some embodiments, the set of nucleotides comprises four nucleobase types, with a first nucleotide having a first nucleobase type and a first label, a second nucleotide having a second nucleobase type and a second label, a third nucleotide having a third nucleobase type and a third label, and a fourth nucleotide having a fourth nucleobase type and an absence of a label, wherein the first label is detectable in the second channel, the second label is detectable in additional channels comprising the third channel, and the third label is detectable in the fourth channel.

In some embodiments, d) comprises identifying an error in the nucleic acid sequencing reaction. In some embodiments, the signal intensity at the first location is measured in: (i) a channel associated with a nucleobase type of a known nucleotide of the control sequence and (ii) one or more channels associated with a different nucleobase type from that of the known nucleotide, and the error is identified based on the signal intensity being greater in one of the one or more channels associated with a different nucleobase type than the channel associated with the nucleobase type of the known nucleotide.

In some embodiments, d) comprises identifying a phasic synchrony error in sequence data captured from the plurality of copies of the control sequence. In some embodiments, the phasic synchrony error comprises a phasing error in the nucleic acid sequencing reaction of the plurality of copies of the control sequence. In some embodiments, the phasic synchrony error comprises a pre-phasing error in the nucleic acid sequencing reaction of the plurality of copies of the control sequence.

In some embodiments, d) comprises determining noise in the signal. In some embodiments, the noise is determined based on a measured signal intensity in a channel associated with a different nucleobase type than the nucleobase type of the known nucleotide of the known sequence. In some embodiments, the method further comprises: e) generating at least one sequence read for the sequence of interest; and f) correcting the sequence read by removing a basecall at a position from a cycle of the sequencing reaction having the error and/or noise identified in d). In some embodiments, removing the basecall comprises assigning an “N” at the position in the position of the cycle having the error and/or noise identified in d). In some embodiments, d) comprises assigning a quality score to the sequence data captured from the plurality of copies of the control sequence based on the comparison between the detected fluorescent signal intensities from the nucleic acid sequencing reaction at the first location to the control sequence. In some embodiments, if the quality score is greater than Q20, the method comprises continuing the nucleic acid sequencing reaction, and if the quality score is less than Q20, the method comprises discarding sequence data from the sequencing reaction.

In some embodiments, d) is performed after each of multiple cycles of the plurality of cycles of the nucleic acid sequencing reaction.

In some embodiments, based on the level of noise and/or error identified in d), the method comprises: e) stripping the control sequencing primer and the second sequencing primer; f) re-hybridizing new molecules of the control sequencing primer to the control sequence; g) performing a second plurality of cycles of a nucleic acid sequencing reaction of the control nucleotide sequence; h) detecting the first fluorescent dye at a location in the biological sample, thereby determining the location of the control sequence in the biological sample; and i) for a cycle of the plurality of cycles at the determined location, comparing a detected fluorescent signal intensity from the nucleic acid sequencing reaction at first location to a known nucleobase of the known sequence, thereby determining a level of noise and/or identifying an error in the nucleic acid sequencing reaction.

In some embodiments, the method comprises performing cycle-by-cycle phasing corrections by determining the presence or absence of a phasing error in d) for each of multiple cycles of the nucleic acid sequencing reaction, and for each of the multiple cycles where a phasing error was identified, calculating a new phasing correction based on the identified phasing error of the respective cycle; and applying the new phasing correction to a subsequent cycle.

In some embodiments, the four nucleobase types are selected from the group consisting of A, T or U, C, and G. In some embodiments, the four nucleobase types are selected from the group consisting of A, T, C, and G.

In some embodiments, the method comprises, prior to step a), contacting the biological sample with a control probe or probe set that binds to a control nucleic acid molecule comprising a complement of the control sequence, and locally amplifying the control probe or probe set to generate a product of the control probe or probe set comprising the plurality of copies of the control sequence. In some embodiments, the control probe or probe set comprises a circularizable probe having ligatable ends, and the method further comprises, prior to the locally amplifying, ligating the ligatable ends to circularize the control probe or probe set. In some embodiments, the method further comprises, prior to step a), contacting the biological sample with an additional probe or probe set that binds to a target of interest, and locally amplifying the additional probe or probe set to generate an amplification product comprising a plurality of copies of the sequence of interest. In some embodiments, the additional probe or probe set comprises a circularizable probe having ligateable ends, and the method further comprises, prior to the locally amplifying, ligating the ligateable ends to circularize the additional probe or probe set.

In some embodiments, the method comprises contacting the biological sample with the control probe or probe set and the additional probe or probe set at a ratio of between about 1:10 and 1:100,000. In some embodiments, the method comprises contacting the biological sample with the control probe or probe set and the additional probe or probe set at a ratio of between about 1:100 and 1:10,000.

In some embodiments, the control nucleic acid molecule in the biological sample comprises RNA. In some embodiments, the control nucleic acid molecule comprises a housekeeping gene transcript in the biological sample. In some embodiments, the housekeeping gene transcript comprises Actin or GapDH.

In some embodiments, the first fluorescent dye is an ultraviolet (UV), deep ultraviolet (DUV) or near ultraviolet (NUV) dye. In some embodiments, the first fluorescent dye is a NUV dye. In some embodiments, the first fluorescent dye is attached to the 5′ end of the control primer. In some embodiments, the first fluorescent dye is attached to the 5′ end of the control primer via a linker.

In some embodiments, the biological sample is a cell sample or tissue sample. In some embodiments, the biological sample comprises a layer of cells deposited on a surface. In some embodiments, the surface is a slide. In some embodiments, the method comprises contacting the biological sample with less than 5 picomoles, less than 1 picomole, or less than 500 femtomoles of the control probe or probe set.

In some aspects, provided herein is a kit for performing nucleic acid sequencing with error correction, comprising: (i) a control sequencing primer labeled with a first fluorescent dye; (ii) a control probe or probe set, wherein the control sequencing primer is capable of binding to a control sequence comprising a known sequence in the control probe or probe set or a product thereof; (iii) a second sequencing primer; (iv) a second probe or probe set, wherein the second probe or probe set is capable of binding to a target of interest, and wherein the second sequencing primer is capable of binding to a sequence of interest in the second probe or probe set or a product thereof; and (v) one or more reagents for performing a nucleic acid sequencing reaction. In some embodiments, the first fluorescent dye is a ultraviolet (UV), deep ultraviolet (DUV) or near ultraviolet (NUV) dye. In some embodiments, the first fluorescent dye is a NUV dye. In some embodiments, the first fluorescent dye is attached to the 5′ end of the control primer. In some embodiments, the first fluorescent dye is attached to the 5′ end of the control primer via a linker.

In some embodiments, the control probe or probe set binds to a housekeeping gene transcript in the biological sample. In some embodiments, the known sequence comprises at least four nucleotides, wherein at least one position of the known sequence is adenine (A), at least one position of the known sequence is thymine (T) or uracil (U), at least one position of the known sequence is guanine (G), and at least one position of the known sequence is cytosine (C). In some embodiments, at least one position of the known sequence is adenine (A), at least one position of the known sequence is thymine (T), at least one position of the known sequence is guanine (G), and at least one position of the known sequence is cytosine (C). In some embodiments, the known sequence comprises a repeating pattern of four nucleobase types. In some embodiments, the known sequence comprises a repeating pattern of four nucleobase types and the known sequence is 12 to 400 nucleotides in length.

In some embodiments, provided herein is a system comprising: one or more processors; and a memory communicatively coupled to the one or more processors and configured to store instructions that, when executed by the one or more processors, cause the system to perform the method of any of the preceding embodiments.

In some aspects, provided herein is a system comprising: a biological sample comprising, at a first location, a rolling circle amplification product comprising a control sequence comprising a known sequence and, at a second location, a rolling circle amplification product comprising a sequence of interest; a control sequencing primer labeled with a first fluorescent dye, wherein the control sequencing primer binds adjacent to the control sequence; and a second sequencing primer, wherein the second sequencing primer binds adjacent to a sequence of interest; a plurality of fluorescently labeled dNTPs that are labeled with fluorescent dyes that emit signals in channels that are different from the first fluorescent dye; and a polymerase.

In some embodiments, the system further comprises an imaging device. In some embodiments, the biological sample is attached to a solid support. In some embodiments, the first fluorescent dye is an ultraviolet (UV), deep ultraviolet (DUV) or near ultraviolet (NUV) dye.

### BRIEF DESCRIPTION OF THE DRAWINGS

The drawings illustrate certain features and advantages of this disclosure. These embodiments are not intended to limit the scope of the appended claims in any manner.

FIG. 1A provides a schematic illustration of an example of a method comprising hybridizing a control probe comprising a complement of a control sequence to a control nucleic acid molecule, generating a rolling circle amplification product (RCP) comprising a plurality of copies of the control sequence, and hybridizing a control sequencing primer to the control sequence.

FIG. 1B provides a schematic illustration of an example of a method comprising hybridizing an additional probe to a target of interest, generating a rolling circle amplification product (RCP) comprising a plurality of copies of a sequence of interest, and hybridizing a second sequencing primer to the sequence of interest.

FIG. 2 depicts a system for performing a sequencing assay, in accordance with some implementations of the methods described herein.

FIG. 3 depicts a computer system or computer network, in accordance with some instances of the systems described herein.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some embodiments, methods for sequencing nucleic acid molecules described herein comprise: a) contacting a biological sample, wherein the biological sample comprises at a first location a plurality of copies of a control sequence comprising a known sequence, with sequencing primers comprising: (i) a control sequencing primer labeled with a first fluorescent dye, wherein the control sequencing primer binds adjacent to the control sequence; and (ii) a second sequencing primer, wherein the second sequencing primer binds adjacent to a sequence of interest; b) detecting the first fluorescent dye at the first location, thereby identifying the first location; c) performing a plurality of cycles of a nucleic acid sequencing reaction by extending the sequencing primers; and d) for a cycle of the plurality of cycles at the identified first location, comparing a signal from the nucleic acid sequencing reaction to an expected signal from the known sequence, thereby determining noise in the signal and/or identifying an error in the nucleic acid sequencing reaction.

In some embodiments, provided herein are methods for sequencing nucleic acid molecules in situ in a biological sample (e.g., a cell or tissue sample). In some embodiments, the biological sample comprises a plurality of copies of a control sequence at a first location, and a plurality of copies of a sequence of interest at another location in the biological sample. In some embodiments, the plurality of copies of the control sequence are in an RCP generated from a control probe or probe set (e.g., a control circularizable probe). In some embodiments, the plurality of copies of the sequence of interest are in an RCP generated from a probe or probe set that binds to a target nucleic acid of interest. In some instances, the target nucleic acid of interest is a nucleic acid analyte in the biological sample. In some instances, the target nucleic acid of interest is a reporter oligonucleotide in a labeling agent that is associated with an analyte in the biological sample (e.g., a non-nucleic acid analyte such as a protein). In some embodiments, the biological sample is contacted with the control probe or probe set and a population of additional probes or probe sets targeting a plurality of different target nucleic acids in the biological sample. In some cases, the control probe or probe set and the additional probes or probe sets are amplified in the biological sample by rolling circle amplification. The resulting RCPs comprise a plurality of copies of the control sequence or of the sequence(s) of interest at locations associated with the target nucleic acid molecules in the biological sample.

During in situ sequencing, sequencing primers are bound adjacent to the copies of sequences of interest and extended to sequence the sequence of interest or a portion of the sequence of interest in a nucleic acid sequencing reaction. In some embodiments, a common primer sequence is present in each of a plurality of sequences of interest, such that a common sequencing primer can be used to sequence the different sequences of interest. In some embodiments, a plurality of sequences of interest comprises a common sequencing primer-binding region and different barcode or marker sequences 5′ of the sequencing primer-binding region. In some embodiments, the marker sequence is a sequence of the target nucleic acid molecule that is targeted by a circularizable probe or probe set. In some embodiments, the complement of the marker sequence is incorporated into a circularizable probe or probe set in a gap-fill reaction (e.g., splint-mediated gap-fill or gap-fill by extension using a polymerase) using the target nucleic acid molecule as a template. Sequencing allows detection of the target nucleic acid molecules at their locations in the biological sample. However, the locations of the target nucleic acid molecules and expected sequences to be detected at particular reactions are not known a priori, making it difficult to monitor accumulations of noise in the nucleic acid sequencing reaction.

In some aspects, the present application addresses the need for nucleic acid sequencing methods with detection of noise and/or sequencing errors. By providing a plurality of copies of a control sequence targeted by a special “control” primer that is tagged with a label identifying the control primer, the location of the control sequence comprising a known sequence can be determined based on detecting the control primer. In some embodiments, the method comprises detecting the label identifying the control primer (e.g., the first fluorescent dye), thereby determining the location of the plurality of copies of the control sequence. Because both the location and the sequence of the control sequence are known, the results of a nucleic acid sequencing reaction at the location of the control sequence can be monitored in real-time. In some embodiments, the method comprises comparing the results of the nucleic acid sequencing reaction at the location of the control sequence to the expected results based on the known sequence, thereby determining a level of noise and/or identifying an error in the sequencing reaction. In some embodiments, the method further comprises adjusting a sequencing algorithm used in subsequent cycles of the nucleic acid sequencing reaction based on the level of noise detected.

In some embodiments, the control sequencing primer is tagged on the 5′ end with an NUV fluorophore. In some embodiments, the control sequence (e.g., in an RCP) comprises a known sequence that is a simple, repetitive sequence of the 4 bases (e.g., A (590), T (532), C (647), and G (dark)). This way, the instrument/pipeline will know a priori what the signal should be for every RCP observed that shows in the NUV channel. Under perfect conditions, with total incorporation and complete cleavage over many cycles, we would observe signal in the channel corresponding to the correct base at each position, and no signal in the other channels. However, no chemical reaction is perfectly complete. In some aspects of the presently provided methods, the degree of incompleteness can now be determined by analyzing the ratio of expected wavelength intensity of signal/cycle against what is observed. Based on this comparison, in some embodiments the method comprises determining the degree of chromatic blur and phasing in the nucleic acid sequencing reaction. In some embodiments, the method comprises adjusting a sequencing algorithm (e.g., adjusting fluorescent signal threshold values used in performing base calls) to improve the accuracy of sequencing of sequencing using the additional sequencing primers, based on the degree of chromatic blur and phasing detected. In some embodiments, if there is some type of dispense error or other malfunction during the run, the method comprises provide precise metrics to a user performing the method about when the issue occurred.

Additional aspects of the methods, compositions, kits, and systems disclosed herein are described in the sections below.

### II. Methods for Nucleic Acid Sequencing with Control Sequences

The sequencing methods described herein are useful for multi-cycle sequencing approaches where nucleotides of nucleotide strand are “interrogated” by binding to a complementary nucleotide. For example, the sequencing methods described herein are applicable to both in situ sequencing applications (e.g., in situ sequencing of endogenous nucleic acid sequences and/or target-specific barcode sequences associated with target analytes of interest that are distributed within a cell or tissue sample) and to more conventional “sequencing in a flow cell” applications (e.g., sequencing of endogenous nucleic acid sequences extracted from a cell or tissue sample). The in situ and flow cell sequencing approaches differ in terms of the sample preparation steps required, as described elsewhere herein, but can share common features in terms of the cyclic series of steps performed to identify nucleotides base-by-base in a template nucleic acid sequence (e.g., a target analyte sequence and/or an associated target-specific barcode sequence). Additionally, the sequencing methods described herein are useful for nucleic acid sequencing reactions including sequencing-by-synthesis, sequencing-by-binding, and sequencing-by-avidity.

### A. Control Sequence and Control Sequencing Primer

In some embodiments, the control sequencing primer hybridizes to a control sequence, and a plurality of cycles of a sequencing reaction are performed to sequence a portion of the control sequence. In some embodiments, a plurality of copies of the control sequence are in a rolling circle amplification product at a location in the biological sample. For example, in some cases a control circularizable probe or probe set is bound directly or indirectly to a target analyte in the biological sample, circularized, and used to generate a rolling circle amplification product comprising the plurality of copies of the control sequence. In some embodiments, the plurality of copies of the control sequence comprise locally amplified copies of the control sequence. In some embodiments, the target analyte for the control probe or probe set is a housekeeping gene. In some cases, a control probe or probe set is not bound to a target analyte in the biological sample. In some embodiments, the control probe or probe set or a rolling circle amplification product thereof is bound to or associated with a matrix embedding the biological sample. In some embodiments, the control circularizable probe or probe set comprises a complement of the control sequence. In some embodiments, the method comprises contacting the biological sample with less than 5 picomoles, less than 1 picomole, or less than 500 femtomoles of the control probe or probe set.

In some embodiments, a plurality of copies of the control sequence are in a plurality of probes that hybridize to a target nucleic acid molecule, such as an mRNA. In some embodiments, the plurality of copies of the control sequence are in a plurality of probes that hybridize to a plurality of different sequences present in a target nucleic acid molecule such as an mRNA (e.g., probes designed to bind the target nucleic acid molecule in a tiled fashion). Binding of a plurality of control probes to the same target nucleic acid molecule thus provides a plurality of copies of the control sequence at the location of the target nucleic acid molecule. In some embodiments, a plurality of copies of the control sequence are generated by clonal amplification (e.g., bridge amplification) in a sequencing flow cell.

The control sequence comprises a region capable of hybridizing to the control sequencing primer (a control primer-binding sequence), and a known sequence (e.g., a known barcode sequence) that can be analyzed by extending the control sequencing primer in a plurality of cycles of a nucleic acid sequencing reaction. In some embodiments, the control primer-binding sequence is complementary to the control sequencing primer. In some embodiments, the control sequencing primer hybridizes specifically to the control-primer binding sequence (e.g., the control sequencing primer is incubated with the biological sample under conditions wherein the control sequencing primer hybridizes specifically to the control-primer binding sequence). In some embodiments, the control primer-binding sequence uniquely identifies the control probe or probe set or a product of the control probe or probe set.

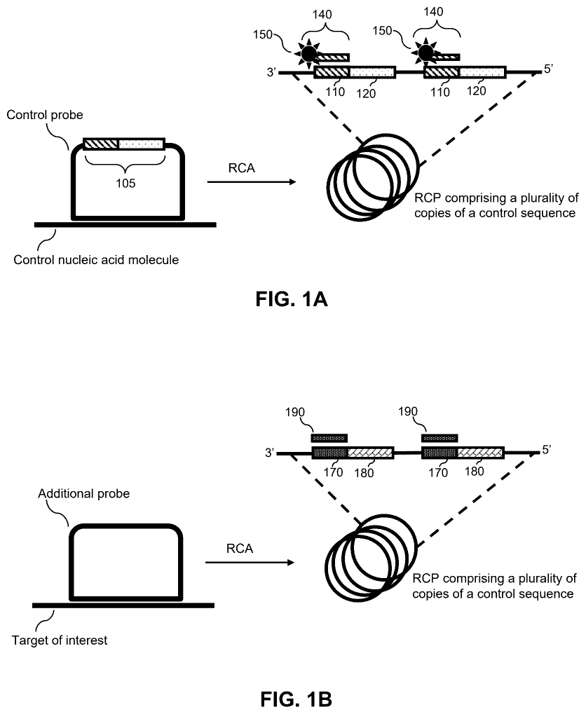

FIG. 1A provides a schematic illustration of a method comprising hybridizing a control probe comprising a complement of a control sequence 105 to a control nucleic acid molecule. In some embodiments, the control nucleic acid molecule is an mRNA of a housekeeping gene. In some embodiments, the control nucleic acid molecule is ubiquitously expressed in cells of the biological sample. In the illustrated embodiments, the control probe is a circular probe or a circularizable probe or probe set that is circularized. The circular or circularized probe is amplified via rolling circle amplification to generate a rolling circle amplification product (RCP) comprising a plurality of copies of the control sequence 120 and the control primer-binding sequence 110. The biological sample is contacted with a control primer 140 comprising a first fluorescent dye 150 that identifies the control primer. In some embodiments, the control probe hybridizes to the control primer-binding sequence in the control sequence, and is extended in a plurality of cycles of a nucleic acid sequencing reaction to sequence all or a portion of the known sequence. In some embodiments, the method comprises detecting the first fluorescent dye 150, thereby identifying the location of the plurality of copies of the control sequence 120 adjacent to the bound control probe in the biological sample. In some embodiments, fluorescent signals detected in each of a plurality of cycles of the nucleic acid sequencing reaction are compared to expected fluorescent signals (e.g., expected fluorescent signal intensities) based on the nucleobase at a corresponding position of the known sequence. For example, if the base A is incorporated during a sequencing cycle, and it is labeled with the fluorescent dye cyanine 5 (Cy5), one would expect a fluorescent signal from Cy5 at that cycle.

In some embodiments, the plurality of cycles comprises at least four cycles. In some embodiments, at least one position of the known sequence is adenine (A), at least one position of the known sequence is thymine (T) or uracil (U), at least one position of the known sequence is guanine (G), and at least one position of the known sequence is cytosine (C). In some embodiments, at least one position of the known sequence is adenine (A), at least one position of the known sequence is thymine (T), at least one position of the known sequence is guanine (G), and at least one position of the known sequence is cytosine (C). In some embodiments, the known sequence is a simple, repetitive sequence of A, T, C, and G (e.g., a repeating pattern of the four different nucleobases, in any order). In some embodiments, the known sequence is a simple, repetitive sequence of A, U, C, and G (e.g., a repeating pattern of the four different nucleobases, in any order). In some embodiments, the known sequence comprises a repeating sequence comprising at least 2, 3, 4, 5, 6, 7, 8, 9, or 10 copies of a four-base unit sequence, wherein the four-base unit sequence comprises one of each of the four nucleobases. In some embodiments, the known sequence is between 12 and 400 nucleotides in length.

In some embodiments, the nucleic acid sequencing reaction comprises at least 15, at least 20, at least 30, or at least 40 cycles of base-by-base sequencing. In some embodiments, the method comprises sequencing at least 5, at least 10, at least 15, at least 20, or at least 30 bases of the known sequence. In some embodiments, the method comprises sequencing no more than 50, no more than 40, no more than 35, no more than 30, or no more than 25 bases of the known sequence.

In some embodiments, a method provided herein comprises contacting a biological sample comprising a plurality of copies of a control sequence at a first location in the biological sample with a control sequencing primer labeled with a first fluorescent dye, wherein the control sequencing primer binds adjacent to the control sequence. In some embodiments, the control

In some embodiments, the first fluorescent dye is an ultraviolet (UV), deep ultraviolet (DUV) or near ultraviolet (NUV) dye. In some embodiments, the first fluorescent dye is a NUV dye. In some embodiments, the first fluorescent dye is attached to the 5′ end of the control primer.

In some embodiments, the first fluorescent dye is attached to the 5′ end of the control primer via a linker. In some embodiments, the linker comprises a cleavable linker. In some embodiments, the cleavable linker comprises a photocleavable linker, a Pd-cleavable linker, a phosphine-cleavable linker, or a disulfide linker.

In some embodiments, a linker comprises a polymer chain. In some embodiments, the polymer chain comprises at least about 2, at least about 5, at least about 8, at least about 10, at least about 12, at least about 15, at least about 20, at least about 25, at least about 50, at least about 75, or at least about 100 monomer units. In some embodiments, the polymer chain is polyethylene glycol, polypropylene glycol, polyvinyl acetate, polylactic acid, or polyglycolic acid. In some embodiments, the polymer chain is a linear or branched molecule. In some embodiments, the linker comprises a polyethylene glycol (PEG) linker. In some embodiments, the PEG linker comprises at least about 2, at least about 5, at least about 8, at least about 10, at least about 12, at least about 15, at least about 20, at least about 25, at least about 50, at least about 75, or at least about 100 ethylene glycol units. In some embodiments, the PEG linker comprises about 2 to about 24 ethylene glycol units.

In some embodiments, a linker comprises a photocleavable linker. Any suitable photocleavable linker can be used (see, e.g., Seo et al. (2005), PNAS 102(17): 5926-5931, incorporated by reference herein in its entirety). In some embodiments, the photocleavable linker comprises a nitrobenzyl group. For instance, a photocleavable nitrobenzyl linker can be cleaved using laser irradiation (355 nm, 10 seconds, 1.5 Wcm⁻²).

In some embodiments, a linker comprises a Pd-cleavable linker. Any suitable Pd-cleavable linker can be used (see, e.g., Ju et al. (2006), PNAS 103(52): 19635-19640, incorporated by reference herein in its entirety). In some embodiments, the Pd-cleavable linker comprises an allyl group. For instance, a Pd-cleavable allyl linker can be cleaved using incubation with a Na₂PdCl₄/P(PhSO₃Na)₃ mixture (30 seconds at 70° C.).

In some embodiments, a linker comprises a phosphine-cleavable linker. Any suitable phosphine-cleavable linker can be used (see, e.g., Guo et al. (2008), PNAS 105(27): 9145-9150, incorporated by reference herein in its entirety). In some embodiments, the phosphine-cleavable linker comprises an azide group. For instance, a phosphine-cleavable azide linker can be cleaved using incubation with a Tris(2-carboxyethyl) phosphine (TCEP) mixture (15 minutes at 65° C.).

In some embodiments, a linker comprises a disulfide bond. For instance, the disulfide bond can be cleaved using incubation with a reducing agent, such as beta-mercaptoethanol, TCEP, or dithiothreitol (DTT).

### B. Sequences of Interest and Sequencing Primers for Sequences of Interest

In some embodiments, the methods provided herein comprise contacting the biological sample with a second sequencing primer, wherein the second sequencing primer binds adjacent to a sequence of interest. In some embodiments, the control primer and the second sequencing primer are both extended in the plurality of cycles of the nucleic acid sequencing reaction.

In some embodiments, the sequence of interest is in an endogenous nucleic acid molecule (e.g., a DNA molecule, an RNA molecule, or an mRNA molecule) that has been reverse transcribed, amplified, and/or extracted from a biological sample (e.g., a cell sample or tissue sample).

In some embodiments, the sequence of interest comprises a barcode sequence (e.g., a nucleic acid barcode sequence) associated with a target analyte of interest (e.g., using the barcoding methods described elsewhere herein), optionally wherein the target analyte has been reverse transcribed, amplified, and/or extracted from a biological sample (e.g., a cell sample or tissue sample).

FIG. 1B provides a schematic illustration of a method comprising hybridizing an additional probe to a target of interest in the biological sample. In the illustrated embodiments, the additional probe is a circular probe or a circularizable probe or probe set that is circularized. The circular or circularized probe is amplified via rolling circle amplification to generate a rolling circle amplification product (RCP) comprising a plurality of copies of a sequencing primer-binding region 170 adjacent to a sequence of interest 180. In some embodiments, the sequence of interest 180 is a sequence to be determined in the nucleic acid sequencing reaction. In some embodiments, the sequence to be determined in the nucleic acid sequencing reaction comprises a barcode sequence. In some embodiments, the sequence to be determined in the nucleic acid sequencing reaction comprises a sequence of the target of interest. In some embodiments, a complement of the sequence 180 to be determined in the nucleic acid sequencing reaction is introduced into the additional circularizable probe or probe set in a gap-fill reaction (e.g., by ligation of a gap-fill splint or gap-fill extension using a polymerase). In some embodiments, the sequence of interest 180 is adjacent to and 3′ of the sequencing primer-binding region 170, wherein the sequencing primer-binding region 170 is complementary to the second sequencing primer.

In some embodiments, the method comprises contacting the biological sample with the control probe or probe set and the additional probe or probe set at a ratio of between about 1:10 and 1:100,000, between about 1:100 and 1:100,000, between about 1:1,000 and 1:100,000, between about 1:10,000 and 1:100,000, between about 1:1,000 and 1:10,000, between about 1:100 and 1:10,000, or between about 1:100 and 1:1,000 (control probe or probe set to additional probe or probe set).

In some embodiments, the target of interest includes a target analyte nucleic acid molecule (e.g., a DNA molecule, an RNA molecule, or an mRNA molecule). In some embodiments, the template nucleic acid includes a reporter oligonucleotide, such as a barcode.

In some embodiments, the target of interest is a DNA molecule. Examples of DNA template nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. In some embodiments, the DNA molecule is copied from another nucleic acid molecule (e.g., DNA or RNA such as mRNA).

In some embodiments, the target of interest an RNA molecule. Examples of RNA template nucleic acid molecules include RNA molecules such as various types of coding and non-coding RNA. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. In some embodiments, the RNA template nucleic acid molecule is copied from another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. In some embodiments, the RNA is small (e.g., less than 200 nucleic acid bases in length) or large (e.g., RNA greater than 200 nucleic acid bases in length). Examples of small RNAs include 5.85 ribosomal RNA (rRNA), 5S rRNA, tRNA, miRNA, siRNA, snoRNAs, piRNA, tRNA-derived small RNA (tsRNA), and small rDNA-derived RNA (srRNA). In some embodiments, the RNA is double-stranded RNA or single-stranded RNA. In some embodiments, the RNA is circular RNA. In some embodiments, the RNA is a bacterial rRNA (e.g., 16s rRNA or 23s rRNA).

In some embodiments, the target of interest comprises a nucleic acid analyte derived from a biological sample and/or a reporter oligonucleotide or nucleic acid marker associated with an analyte from a biological sample. Such analytes can be or derived from any biological sample. In some embodiments, the template nucleic acid comprises a nucleic acid analyte and/or a reporter oligonucleotide or nucleic acid marker associated with an analyte present in a biological sample, and the template nucleic acid molecule is sequenced at a location in the biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, in some embodiments, a biological sample is obtained from a prokaryote such as a bacterium, an archaea, a virus, or a viroid. In some embodiments, a biological sample is obtained from non-mammalian organisms (e.g., a plant, an insect, an arachnid, a nematode, a fungus, or an amphibian). In some embodiments, a biological sample is obtained from a eukaryote, such as a tissue sample, a patient derived organoid (PDO) or patient derived xenograft (PDX). In some embodiments, a biological sample from an organism comprises one or more other organisms or components therefrom. For example, a mammalian tissue section may comprise a prion, a viroid, a virus, a bacterium, a fungus, or components from other organisms, in addition to mammalian cells and non-cellular tissue components. In some embodiments, subjects from which biological samples are obtained are healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, a target of interest includes a reporter oligonucleotide or marker associated with the presence of an analyte (e.g., an endogenous analyte) in a sample. Such analytes may include nucleic acid analytes and/or non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In some embodiments, a target of interest is a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, methods and compositions disclosed herein are used to analyze any number of targets of interest (e.g., nucleic acid analytes and/or analyte-associated barcode sequences) or fragments thereof. For example, in some embodiments, the number of analytes that are analyzed is at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of a sample (e.g., a cell sample or tissue sample) or tethered within individual features on a substrate (e.g., a flow cell surface). Targets of interest are further described elsewhere herein (e.g., analytes, labeling agents, and/or hybridization, ligation, or polymerase extension products in the biological sample).

C. Nucleic Acid Sequencing Reactions and Noise and/or Error Detection

In some embodiments, the present application provides methods for nucleic acid sequencing wherein noise and/or sequencing errors can be detected and accounted for in subsequent cycles of the nucleic acid sequencing reaction. In some embodiments, the noise and/or sequencing errors are identified based on sequencing a known sequence within a plurality of copies of a control sequence, wherein the plurality of copies of the control sequence are localized at a first location of the biological sample. In some embodiments, the first location is unambiguously identified by detected a detectable label associated with a control sequencing primer in the biological sample, wherein the control sequencing primer hybridizes to the control sequence. In some embodiments, the control sequencing primer is used to prime a nucleic acid sequencing reaction of the known sequence. At the same time, one or more additional sequencing primers is/are used to sequence one or more sequences of interest at other locations in the biological sample. Based on detected signal intensities at the first location, a level of noise and/or a sequencing error is determined and used to correct or adjust the sequencing algorithm or results for the sequences of interest at other locations in the biological sample.

In some embodiments, each cycle of the nucleic acid sequencing reaction comprises determining a fluorescent signal intensity indicative of incorporation or binding of a nucleotide of a particular nucleobase type at one or more locations comprising the location of a plurality of copies of the control sequence. In some embodiments, a portion of the detected fluorescent signal intensity is noise. In some embodiments, the noise is noise associated with a nucleotide incorporated during a previous cycle of the sequencing reaction (e.g., due to incomplete cleavage of a fluorophore from a previously incorporated nucleotide). In some embodiments, the method comprises comparing the detected fluorescent signal intensity at the first location in the biological sample (the location of the plurality of copies of the control sequence) to an expected fluorescent signal intensity for a corresponding position of the known sequence, thereby determining the portion of the detected signal intensity that is noise.

In some embodiments, the method comprises correcting the fluorescent signal intensity value by subtracting the noise. In some embodiments, the method comprises correcting the fluorescent signal intensity value by subtracting the noise for each of a plurality of cycles of the nucleic acid sequencing reaction. In some embodiments, a new level of noise is calculated for each of a plurality of cycles of the nucleic acid sequencing reaction. In some embodiments, a new level of noise is calculated for each cycle of the nucleic acid sequencing reaction.

In some embodiments, the method comprises adjusting a base calling algorithm used in the nucleic acid sequencing reaction in order to account for the noise. In some instances, adjusting the base calling algorithm comprises adjusting a set of threshold fluorescent signal intensity values used to infer a base call. In some embodiments, a new set of threshold fluorescent signal intensity values used to infer a base call is calculated for each of a plurality of cycles of the nucleic acid sequencing reaction. In some embodiments, a new set of threshold fluorescent signal intensity values used to infer a base call is calculated for each cycle of the nucleic acid sequencing reaction.

In some embodiments, the method comprises comparing the detected fluorescent signal intensity at the first location in the biological sample (the location of the plurality of copies of the control sequence) to an expected fluorescent signal intensity for a corresponding position of the known sequence, thereby evaluating the quality of a base call in the nucleic acid sequencing reaction. Accordingly, presented herein are methods and systems for evaluating the quality of a base call from a sequencing read. In some embodiments, the method comprises determining a quality score for a base call based on the comparison of the detected fluorescent signal intensity at the first location in the biological sample to the expected fluorescent signal intensity for a corresponding position of the known sequence. The quality score is typically quoted as QXX where the XX is the score and it means that that particular call has a probability of error of 10(−XX/10). For example Q30 equates to an error rate of 1 in 1000, or 0.1% and Q40 equates to an error rate of 1 in 10,000 or 0.01%. In some embodiments, after calculating quality scores, the method further comprises discounting base calls with unreliable quality scores. In some embodiments, the method further comprises: e) generating at least one sequence read for the sequence of interest; and f) correcting the sequence read by removing a basecall at a position from a cycle of the sequencing reaction having the error and/or noise identified in d). In some embodiments, removing the basecall comprises assigning an “N” at the position in the position of the cycle having the error and/or noise identified in d). In some embodiments, d) comprises assigning a quality score to the sequence data captured from the plurality of copies of the control sequence based on the comparison between the detected fluorescent signal intensities from the nucleic acid sequencing reaction at the first location to the control sequence. In some embodiments, if the quality score is greater than Q20, the method comprises continuing the nucleic acid sequencing reaction, and if the quality score is less than Q20, the method comprises discarding sequence data from the sequencing reaction.

In some embodiments, the method comprises identifying an error based on the signal intensity detected at the first location in the biological sample. In some embodiments, the signal intensity at the first location is measured in: (i) a channel associated with a nucleobase type of a known nucleotide of the control sequence and (ii) one or more channels associated with a different nucleobase type from that of the known nucleotide, and the error is identified based on the signal intensity being greater in one of the one or more channels associated with a different nucleobase type than the channel associated with the nucleobase type of the known nucleotide.

In some embodiments, the method comprises identifying a phasic synchrony error in sequence data captured from the plurality of copies of the control sequence. In some embodiments, the phasic synchrony error comprises a phasing error in the nucleic acid sequencing reaction of the plurality of copies of the control sequence. In some embodiments, the phasic synchrony error comprises a pre-phasing error in the nucleic acid sequencing reaction of the plurality of copies of the control sequence.

In some embodiments the first fluorescent dye of the control sequencing primer is detected in order to identify the location of the control sequence independently from analyzing the known barcode sequence of the control sequence. The first fluorescent dye can be detected before the nucleic acid sequencing reaction, and/or during the nucleic acid sequencing reaction. In some embodiments, the method comprises performing a plurality of cycles of a nucleic acid sequencing reaction by extending the sequencing primers before detecting the first fluorescent dye at the first location, thereby identifying the first location. In some embodiments, detecting the first fluorescent dye at the first location is performed prior to, simultaneously with, or subsequent to the plurality of cycles of the nucleic acid sequencing reaction. In some embodiments, the first fluorescent dye at the first location is detected in multiple cycles of the sequencing reaction. In some cases, the first fluorescent dye is distinguishable from any of the labels used to label nucleotides comprising different nucleobases in the nucleic acid sequencing reaction. In some embodiments, the first fluorescent dye at the first location is detected before a plurality of cycles of the sequencing reaction. In some embodiments, the method comprises cleaving the first fluorescent dye from the control sequencing primer or quenching the first fluorescent dye before performing the plurality of cycles of the sequencing reaction. In some cases, the first fluorescent dye has an emission spectrum that overlaps with the emission spectrum of one of the labels used to label nucleotides comprising different nucleobases in the nucleic acid sequencing reaction, and the first fluorescent dye is cleaved or quenched prior to performing the plurality of cycles of the sequencing reaction.

In some embodiments, based on the level of noise and/or error identified in d), the method comprises: e) stripping the control sequencing primer and the second sequencing primer; f) re-hybridizing new molecules of the control sequencing primer to the control sequence; g) performing a second plurality of cycles of a nucleic acid sequencing reaction of the control nucleotide sequence; h) detecting the first fluorescent dye at a location in the biological sample, thereby determining the location of the control sequence in the biological sample; and i) for a cycle of the plurality of cycles at the determined location, comparing a detected fluorescent signal intensity from the nucleic acid sequencing reaction at first location to a known nucleobase of the known sequence, thereby determining a level of noise and/or identifying an error in the nucleic acid sequencing reaction.

In some embodiments, the method comprises performing cycle-by-cycle phasing corrections by determining the presence or absence of a phasing error in d) for each of multiple cycles of the nucleic acid sequencing reaction, and for each of the multiple cycles where a phasing error was identified, calculating a new phasing correction based on the identified phasing error of the respective cycle; and applying the new phasing correction to a subsequent cycle. In some embodiments, phasing errors occur when a sequencing reaction at a location falls at least one base behind other sequencing reactions at the same optically resolved location. For example, incomplete cleavage of a reversible terminator at a sequencing reaction for one copy of a plurality of copies of a control sequence results in accumulation of noise in the detected signal intensities due to phasing. In some embodiments, the phasing error comprises pre-phasing. In some embodiments, pre-phasing occurs when a nucleic acid sequencing reaction at a location jumps at least one base ahead of other nucleic acid sequencing reactions at the same optically resolved location. For example, incomplete cleavage of a sequencing reaction for one copy of a plurality of copies of a control sequence may jump ahead of sequencing reactions at other copies of the plurality of copies of the control sequence. The effects of phasing and pre-phasing become more pronounced with higher phasing/prephasing rates and longer reads. Thus, in order to maintain accurate base calling over an extended number of cycles, it is important to correct for this phenomenon. The methods and systems presented herein provide a solution for improved base calling over extended sequencing cycles by adjusting for phasing errors in each of a plurality of cycles using the known sequence of the control sequence.

In some embodiments, the methods and systems provided herein can assume that a fixed fraction of molecules at each location become phased at each cycle, in the sense that those molecules fall one base behind in sequencing. In some embodiments, the phasing correction and/or algorithm adjustment determined based on sequencing the control sequence is applied to the sequencing reaction for one or more sequences of interest being simultaneously sequenced in the biological sample.

In some embodiments, the first fluorescent dye is detectable in a first channel, and each cycle of the nucleic acid sequencing reaction comprises detecting a fluorescent signal intensity indicative of incorporation or binding of a nucleotide of a particular nucleobase type in additional channels comprising a second channel and a third channel. In some embodiments, the nucleic acid sequencing reaction is performed using 2-color sequencing chemistry. In some embodiments, for the 2-color chemistry nucleic acid sequencing reaction, the set of nucleotides comprises first nucleotides having a first nucleobase type and a first label, a second nucleotides having a second nucleobase type and a second label, a mixture of third nucleotides having a third nucleobase type wherein 40-60% of the mixture has the first label and the remainder of the mixture has the second label, and a fourth nucleotide having a fourth nucleobase type and an absence of a label, and the first label is detectable in the second channel and a second label is detectable in the third channel. In some embodiments, the first and second labels are not detectable or are minimally detectable in the first channel.

In some embodiments, for the 2-color chemistry, an expected signal for the first nucleobase type comprises a greater signal intensity in the second channel than the third channel, an expected signal for the second nucleobase type is a greater signal intensity in the third channel than the second channel, an expected signal for the third nucleobase type is a signal intensity in the second channel that is 90%-110% of the signal intensity in the third channel, and an expected signal intensity for the fourth nucleobase type is a minimal signal in the second channel and third channel. In some embodiments, d) comprises identifying an error, and for a sequenced position of the known sequence where a nucleobase type is known, the measured signal intensity in the second and third channels does not match an expected signal for the known nucleobase type of the position.

In some embodiments, for the 2-color chemistry, each cycle of the plurality of cycles of step c) comprises measuring an intensity value in each of: the second channel and the third channel, and the method further comprises generating one or more sequence reads for the sequence of interest at a location in the biological sample, wherein a nucleotide is assigned for each position of the sequence read as follows:
(i) the first nucleotide is assigned at the position if an intensity of at least a threshold value is measured in the second channel and intensity of below a cutoff value is measured in the third channel,
(ii) the second nucleotide is assigned at the position if an intensity of at least a threshold value is measured in the third channel and intensity of below a cutoff value is measured in the second channel.
(iii) the third nucleotide is assigned at the position if an intensity of at least a threshold value is measured in the second and third channels,
(iv) the fourth nucleotide is assigned at the position if an intensity of no more than a cutoff value is measured in the second and the third channels, and
(v) an unknown nucleotide (“N”) is assigned at the position if the criteria of none of (i)-(iv) is met. In some embodiments, the threshold values and cutoff values are determined based on the noise detected in the sequencing reactions for the control sequence.

In some embodiments, the first fluorescent dye is detectable in a first channel, and each cycle of the nucleic acid sequencing reaction comprises detecting a fluorescent signal intensity indicative of incorporation or binding of a nucleotide of a particular nucleobase type in additional channels comprising a second channel, a third channel, and a fourth channel. In some embodiments, the nucleic acid sequencing reaction is performed using 3-color sequencing chemistry. In some embodiments, for the 3-color chemistry nucleic acid sequencing reaction, the set of nucleotides comprises four nucleobase types, with a first nucleotide having a first nucleobase type and a first label, a second nucleotide having a second nucleobase type and a second label, a third nucleotide having a third nucleobase type and a third label, and a fourth nucleotide having a fourth nucleobase type and an absence of a label, wherein the first label is detectable in the second channel, the second label is detectable in additional channels comprising the third channel, and the third label is detectable in the fourth channel.

In some embodiments, for the 3-color chemistry, an expected signal for the first nucleobase type comprises a greater signal intensity in the second channel than the third and fourth channels, an expected signal for the second nucleobase type is a greater signal intensity in the third channel than the second channel and fourth channels, an expected signal for the third nucleobase type is a greater signal in the fourth channel than the second and third channels, and an expected signal intensity for the fourth nucleobase type is a minimal signal in the second, third, and fourth channels. In some embodiments, d) comprises identifying an error, and for a sequenced position of the known sequence where a nucleobase type is known, the measured signal intensity in the second, third, and fourth channels does not match an expected signal for the known nucleobase type of the position.

In some embodiments, for the 3-color chemistry, each cycle of the plurality of cycles of step c) comprises measuring an intensity value in each of: the second channel, the third channel, and the fourth channel, and the method further comprises generating one or more sequence reads for the sequence of interest at a location in the biological sample, wherein a nucleotide is assigned for each position of the sequence read as follows:
(i) the first nucleotide is assigned at the position if an intensity of at least a threshold value is measured in the second channel and intensities of below a cutoff value are measured in the third and the fourth channels,
(ii) the second nucleotide is assigned at the position if an intensity of at least a threshold value is measured in the third channel and intensities of below a cutoff value are measured in the second and the fourth channels.
(iii) the third nucleotide is assigned at the position if an intensity of at least a threshold value is measured in the fourth channel and intensities of below a cutoff value are measured in the second and fourth channels,
(iv) the fourth nucleotide is assigned at the position if an intensity of no more than a cutoff value is measured in all of the second, the third, and the fourth channels, and
(v) an unknown nucleotide (“N”) is assigned at the position if the criteria of none of (i)-(iv) is met. In some embodiments, the threshold values and cutoff values are determined based on the noise detected in the sequencing reactions for the control sequence.

In some embodiments, the nucleic acid sequencing reaction is performed using 4-color sequencing chemistry. In some embodiments, for the 4-color chemistry nucleic acid sequencing reaction, the set of nucleotides comprises first nucleotides having a first nucleobase type and a first label, a second nucleotides having a second nucleobase type and a second label, a third nucleotide having a third nucleobase type and a third label, and a fourth nucleotide having a fourth nucleobase type and a fourth label. In some embodiments, the first fluorescent dye is detectable in a first channel, and each cycle of the nucleic acid sequencing reaction comprises detecting a fluorescent signal intensity indicative of incorporation or binding of a nucleotide of a particular nucleobase type in additional channels comprising a second channel, a third channel, a fourth channel, and a fifth channel. In some embodiments, the first label is detectable in the second channel, the second label is detectable in the third channel, the third label is detectable in the fourth channel, and the fourth label is detectable in the fifth channel. In some embodiments, the first fluorescent dye is detectable in a first channel, the method comprises cleaving or quenching the first fluorescent dye before performing the cycles of the nucleic acid sequencing reaction, and each cycle of the nucleic acid sequencing reaction comprises detecting a fluorescent signal intensity indicative of incorporation or binding of a nucleotide of a particular nucleobase type in the first channel and additional channels comprising a second channel, a third channel, and a fourth channel. In some embodiments, the first label is detectable in the first channel, the second label is detectable in the second channel, the third label is detectable in the third channel, and the fourth label is detectable in the fourth channel.

In some embodiments, for the 4-color chemistry, each cycle of the plurality of cycles of step c) comprises measuring an intensity value in each of: the second channel, the third channel, the fourth channel, and the fifth channel, and the method further comprises generating one or more sequence reads for the sequence of interest at a location in the biological sample, wherein a nucleotide is assigned for each position of the sequence read as follows:
(i) the first nucleotide is assigned at the position if an intensity of at least a threshold value is measured in the second channel and intensities of below a cutoff value are measured in the third, fourth, and fifth channels,
(ii) the second nucleotide is assigned at the position if an intensity of at least a threshold value is measured in the third channel and intensities of below a cutoff value are measured in the second, fourth, and fifth channels.
(iii) the third nucleotide is assigned at the position if an intensity of at least a threshold value is measured in the fourth channel and intensities of below a cutoff value are measured in the second, third, and fifth channels,
(iv) the fourth nucleotide is assigned at the position if an intensity an intensity of at least a threshold value is measured in the fifth channel and intensities of below a cutoff value are measured in the second, third, and fourth channels, and
(v) an unknown nucleotide (“N”) is assigned at the position if the criteria of none of (i)-(iv) is met. In some embodiments, the threshold values and cutoff values are determined based on the noise detected in the sequencing reactions for the control sequence.

In some embodiments, the disclosed methods further comprise processing optical signals (e.g., fluorescence signals) detected in images (e.g., fluorescence images) acquired during the cyclic series of base-by-base sequencing reactions to detect the presence or absence of complementary nucleotides in the extended priming strand in each sequencing cycle at the locations of each of a plurality of template nucleic acid molecules (i.e., the locations corresponding to each of a plurality of target analyte molecules and/or their associated target-specific barcode sequences), thereby enabling inference of the nucleotide sequence of the plurality of template nucleic acid molecules (e.g., the plurality of target analyte molecules and/or associated target-specific barcode sequences).

The disclosed sequencing methods may be applied to both in situ sequencing and flow cell sequencing applications, where the sequencing reactions are substituted for the stepwise nucleotide incorporation reactions used to probe a template nucleic acid sequence in, e.g., a conventional sequencing-by-synthesis (SBS) method.

In the case of in situ sequencing, in some embodiments, the disclosed methods comprise performing all or a subset of the following steps (in addition to a cyclic series of base-by-base sequencing reactions):
(i) preparing the biological sample (e.g., by fixing, sectioning, embedding, and/or clearing a cell or tissue sample, as described elsewhere herein).
(ii) contacting target analytes (e.g., target nucleic acid analytes and/or protein analytes) within the prepared sample with target-specific probes, as described elsewhere herein. In some embodiments, the target-specific probes comprise, e.g., target-specific linear and/or circularizable nucleic acid probes (e.g., padlock probes) designed to hybridize directly or indirectly to specific target nucleic acid analytes. In some embodiments, the target-specific linear and/or circularizable nucleic acid probes comprise primer binding sites and/or target-specific barcode (or identifier) sequences. In some embodiments, the target-specific probes comprise, e.g., target-specific antibodies designed to bind to specific target protein analytes, where the antibodies are conjugated to nucleic acid sequences. In some embodiments, the conjugated nucleic acid sequences comprise primer binding sites and/or target-specific barcode (or identifier) sequences.
(iii) optionally performing a reverse transcription reaction (e.g., if the probed target nucleic acid analytes comprise RNA molecules) to create cDNA copies of RNA target molecules.
(iv) optionally amplifying the probed target analyte molecules and/or their associated target-specific barcode sequences (e.g., using rolling circle amplification (RCA) in the case that target-specific circularizable probes were used to probe target analyte molecules and/or associated barcode sequences).
(v) contacting the optionally amplified target nucleic acid analytes and/or associated target-specific barcode sequences with sequencing primers

In the case of flow cell sequencing, in some embodiments, the disclosed methods comprise performing all or a subset of the following steps (in addition to a cyclic series of base-by-base sequencing reactions):
(i) extraction and purification of nucleic acid molecules (e.g., endogenous nucleic acid sequences) from a biological sample, as described elsewhere herein.
(ii) preparation of a sequencing library comprising template nucleic acid molecules (e.g., the endogenous nucleic acid sequences or fragments thereof) that have been end-repaired and ligated to adapter sequences, as described elsewhere herein.
(iii) optionally performing nucleic acid amplification of all or a portion of the sequencing library, as described elsewhere herein.
(iv) immobilizing the template nucleic acid molecules (e.g., denatured, single-stranded template nucleic acid molecules) from the sequencing library on an inner surface of a flow cell using capture probes (e.g., complementary adapter sequences) that have been tethered to the flow cell surface.
(v) performing clonal amplification of the immobilized template nucleic acid molecules to create clusters comprising, e.g., thousands or tens of thousands of copies of the template nucleic acid molecule immobilized at each of a plurality of locations on the flow cells surface.
(vi) contacting the template nucleic acid molecules in each clonally-amplified cluster with sequencing primers designed to hybridize to, e.g., the adapter sequences ligated to the template nucleic acid molecules. In some embodiments, the sequencing primers comprise free 3′-hydroxyl groups at their 3′ termini, and a primer extension reaction may be performed to incorporate a single nucleotide at the 3′ termini of a bound primer (i.e., the 3′ termini of the priming strands).

In some embodiments, the cyclic series of base-by-base sequencing reactions comprises performing at least 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, or more than 50 cycles of the base-by-base sequencing reaction.

In some embodiments, each cycle of base-by-base sequencing further comprises a first wash step following the contacting step to remove unbound polymerase and nucleotides. In some embodiments, the first wash step comprises, for example, use of the same buffer used for contacting the primed template nucleic acid with a polymerase and a composition nucleotides (but without the polymerase and composition). In some embodiments, the first wash buffer may not include KCl and/or may include little to no DMSO. In some embodiments, the first wash buffer is similar to those used for wash buffers as used in wash steps of a Western blot (e.g., a wash buffer added in a Western blot after binding a primary antibody but washing prior to incubation with a secondary antibody, such as PBST). PBST is a phosphate-buffered saline with a low-concentration of detergent, such as 0.05% to 0.1% Tween.

In some embodiments, each cycle of base-by-base sequencing further comprises photobleaching the fluorophore of the incorporated nucleotide following the detection step. In some embodiments, fluorophore of the incorporated nucleotide is unable to fluoresce after photobleaching. Any suitable photobleaching methods can be implemented. In some embodiments, the sample is exposed to a light source until the signal emitted by the fluorophore is eliminated.

In some embodiments, the nucleotide comprises a reversible terminator moiety. In some embodiments, each cycle of base-by-base sequencing further comprises cleaving the reversible terminator moiety after detecting the incorporated nucleotide.

### Base-Calling

As noted elsewhere herein, the disclosed methods for performing nucleic acid sequencing (e.g., in situ and/or flow cell sequencing) may comprise inferring the sequence of a template nucleic acid molecule from a series of optical signals (e.g., fluorescence signals) detected in images acquired during a repetitive series of sequencing reaction cycles in a process referred to as “base-calling.” The interplay of sequencing chemistry, opto-fluidics hardware, optical sensors, and signal processing software utilized in sequencing platforms affects the types of errors made during sequencing (see, e.g., Lederberger et al. (2011), “Base-calling for next-generation sequencing platforms”, Brief Bioinform. 12(5): 489-497). The characterization of errors associated with the sequencing process and implementation of chemistry-, imaging-, and/or signal processing software-based methods for minimizing sequence errors are thus important for maximizing the accuracy of sequencing results.

In some embodiments, a base call is made based on a set of threshold or cutoff expected fluorescent signal intensities for a given base. For example, if A is labeled with a red fluorescent channel, a base call for A is made based on the fluorescent intensity in the red channel being above a threshold value and the fluorescent intensity in one or more other channels corresponding to other nucleobases being below one or more cutoff values. In some embodiments, the methods provided herein comprise adjusting a base calling algorithm (e.g., adjusting the threshold and/or cutoff values used in base calling) based on a level of noise detected for the control sequence.

In four-color sequencing-by-synthesis methods, for example, a set of four images—one image for each of four detection channels corresponding to the emission wavelengths for four fluorophores used to label the reversibly terminated nucleotides—are acquired in each sequencing cycle. Processing of the images to detect fluorescence intensity signals produces an intensity quadruple for the location of each sequencing colony on a flow cell surface (or the location of each target analyte, or amplified representation thereof (e.g., an RCP) in the case of in situ sequencing), where each value represents the intensity of the fluorescence signal for the detection channels corresponding to A, C, G and T. Ideally, the channel in which the maximum intensity occurs would be the base that is “called” for a given RCP or sequencing colony (or target analyte) in a given cycle. However, the chemical processes involved in sequencing are imperfect, leading to errors in base-calling (see, e.g., Cacho, et al. (2016), “A Comparison of Base-calling Algorithms for Illumina Sequencing Technology”, Briefings in Bioinformatics 17(5):786-795). In some sequencing-by-synthesis (SBS) platforms, for example, sources of error may include phasing (or lagging; e.g., where the primed template nucleic acid molecules at one or more locations fail to incorporate the next base due to variation in polymerase reaction kinetics), pre-phasing (or leading; e.g., where more than one nucleotide is incorporated in a single cycle due to, e.g., impurities in the reversibly terminated nucleotides), signal decay (due to, e.g., photobleaching and/or loss of template nucleic acid during the sequencing process), and cross-talk (e.g., when two or more fluorophore emission spectra overlap, which may cause a positive correlation between signal intensities measured in the corresponding detection channels).

### Sequence Reads

The output of the base-calling process applied to optical signals detected in a series of images of a biological sample or flow cell surface acquired during a cycling sequencing process consists of a plurality of sequence reads, e.g., the nucleotide sequences determined for all or a portion of a template nucleic acid molecule (e.g., an endogenous nucleic acid analyte or a barcode sequence associated with a target analyte).

In some instances, the sequence reads generated using the disclosed methods for in situ and/or flow cell sequencing may comprise sequence reads of at least about 10, 20, 30, 40, 50, 60, 70, 80, 90, 100 or more nucleotides or base pairs of the template nucleic acid sequences. In some instances, the sequence reads generated using the disclosed methods may comprise sequence reads of at least about 100, 120, 140, 160, 180, 200, 220, 240, 260, 280, 300, 320, 340, 360, 380, 400, or more than 400 nucleotides or base pairs of the template nucleic acid sequences.

In some instances, the disclosed methods for in situ or flow cell sequencing may generate at least about 100, 200, 300, 400, 500, 600, 700, 800, 900, 1,000, or more sequencing reads per run. In some instances, the disclosed method may generate at least about 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 10⁶, 5×10⁶, 10⁷, or more than 10⁷ sequencing reads per run.

### Sequence Assembly & Alignment

In some instances, the disclosed methods for in situ and/or flow cell sequencing may comprise assembly of longer template nucleic acid sequences, e.g., genome fragments or whole genomes, from a plurality of relatively short sequence reads. Sequence assembly may be performed by identifying the overlapping sequences from multiple short sequence reads to assemble longer, contiguous sections of sequence.

In some instances, the disclosed methods for in situ and/or flow cell sequencing may comprise identifying a code word corresponding to a sequence read or an assembled sequence, where the code word is one of a plurality of code words in a codebook that includes assignment of each of the plurality of code words to a target analyte of interest. The sequence read or assembled sequence may thus be used to identify a specific target analyte (based on the corresponding code word) in, e.g., a multiplexed in situ detection or sequencing assay.

In some instances, the disclosed methods for in situ and/or flow cell sequencing may comprise alignment of sequence reads and/or assembled sequences to a known reference sequence or consensus sequence (e.g., the GRCh38 human reference genome (Genome Reference Consortium)) from the same or a similar organism. Alignment to a reference sequence or consensus sequence may be used to identify gaps, errors, or variants in the assembled sequence. Any of a variety of available bioinformatics software programs may be used to assemble longer sequences from relatively short sequence reads. Examples include, but are not limited to, DBG2OLC (see, e.g., Ye et al. (2016), “DBG2OLC: Efficient Assembly of Large Genomes Using Long Erroneous Reads of the Third Generation Sequencing Technologies”, Scientific Reports 6:31900), SPAdes (see, e.g., Bankevich et al. (2012), “SPAdes: A New Genome Assembly Algorithm and Its Applications to Single-Cell Sequencing”, J. Computational Biol. 19(5):455-477), SparseAssembler (see, e.g., Ye et al. (2012), “Exploiting Sparseness in de novo Genome Assembly”, BMC Bioinformatics 13(Suppl 6):S1), Fermi (see, e.g., Li (2012), “Exploring Single-Sample SNP and INDEL Calling with Whole-Genome de novo Assembly”, Bioinformatics 28(14):1838-1844), and String Graph Assembler (SGA) (see, e.g., Simpson et al. (2012), “Efficient de novo Assembly of Large Genomes Using Compressed Data Structures”, Genome Res. 22: 549-556), all of which are hereby incorporated by reference herein in their entireties.

In some embodiments, the detection step comprises the use of an optical imaging technique (e.g., a fluorescence imaging technique) and real time or post-processing measurement of optical signals (e.g., fluorescence signals or the absence thereof) associated with the presence of a specific nucleotide at a plurality of locations corresponding to a plurality of target analytes distributed throughout the biological sample or tethered to specific locations on a substrate surface (e.g., a flow cell surface).

### Nucleic Acid Sequencing Reactions

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

A variety of statistical approaches have been developed to correct for, or minimize, such errors and generate more accurate base-calls. Examples include, but are not limited to, AYB (Goldman Group, European Molecular Biology Laboratory—European Bioinformatics Institute, Cambridgeshire, UK), and Bustard (Illumina, Inc., San Diego, CA).

In some embodiments, the polymerase comprises, e.g., Taq polymerase, Therminator™ DNA polymerase, a Klenow fragment of DNA polymerase I, or any combination thereof. In some embodiments, the polymerase is not labeled with a detectable label. Examples of polymerases that are used for performing the disclosed methods include, but are not limited to, DNA polymerases (e.g., Taq DNA polymerase), RNA polymerases, and/or reverse transcriptases.

In some embodiments, the polymerase is a DNA polymerase. Examples of DNA polymerases include Taq polymerase, 9° N-7 DNA polymerase (or variants thereof, for example, D141A/E143A/A485L), phi29 (φ29) polymerase, Klenow fragment, Bacillus stearothermophilus DNA polymerase (BST), T4 DNA polymerase, T7 DNA polymerase, and DNA polymerase I. In some embodiments, DNA polymerases that have been engineered or mutated to have desirable characteristics are employed. In some embodiments, the polymerase is phi29 DNA polymerase. In some embodiments, the polymerase is a DNA polymerase and the template nucleic acid molecule includes DNA. In some embodiments, the polymerase is a DNA polymerase and the nucleotide molecules include deoxyribonucleotides.

In some embodiments, the DNA polymerase is Taq polymerase or a functional variant thereof. Taq polymerase is a heat stable polymerase from Thermus aquaticus. An example Taq polymerase sequence is:

(SEQ ID NO: 1)
GMLPLFEPKGRVLLVDGHHLAYRTFHALKGLTTSRGEPVQAVYGFAKSL
LKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLL
SDRIHVLHPEGYLITPAWLWEKYGLRPDQWADYRALTGDESDNLPGVKG
IGEKTARKLLEEWGSLEALLKNLDRLKPAIREKILAHMDDLKLSWDLAK
VRTDLPLEVDFAKRREPDRERLXAFLERLEFGSLLHEFGLLESPKXLXE
APWPPPERAFVP.

In some embodiments, the DNA polymerase is phi29 DNA polymerase or a functional variant thereof. The DNA polymerase of phi29 (a phage of Bacillus subtilis) has high processivity and fidelity. An example phi29 DNA polymerase sequence is:

(SEQ ID NO: 2)
MKHMPRKMYSCDFETTTKVEDCRVWAYGYMNIEDHSEYKIGNSLDEFMA
WVLKVQADLYFHNLKFDGAFIINWLERNGFKWSADGLPNTYNTIISRMG
QWYMIDICLGYKGKRKIHTVIYDSLKKLPFPVKKIAKDFKLTVLKGDID
YHKERPVGYKITPEEYAYIKNDIQIIAEALLIQFKQGLDRMTAGSDSLK
GFKDIITTKKFKKVFPTLSLGLDKEVRYAYRGGFTWLNDRFKEKEIGEG
MVFDVNSLYPAQMYSRLLPYGEPIVFEGKYVWDEDYPLHIQHIRCEFEL
KEGYIPTIQIKRSRFYKGNEYLKSSGGEIADLWLSNVDLELMKEHYDLY
NVEYISGLKFKATTGLFKDFIDKWTYIKTTSEGAIKQLAKLMLNSLYGK
FASNPDVTGKVPYLKENGALGFRLGEEETKDPVYTPMGVFITAWARYTT
ITAAQACYDRIIYCDTDSIHLTGTEIPDVIKDIVDPKKLGYWAHESTFK
RAKYLRQKTYIQDIYMKEVDGKLVEGSPDDYTDIKFSVKCAGMTDKIKK
EVTFENFKVGFSRKMKPKPVQVPGGVVLVDDTFTIK.

In some embodiments, the DNA polymerase is a 9° N-7 DNA polymerase or a functional variant thereof (e.g., D141A/E143A/A485L). 9° N-7 is a strain of Thermococcus sp. An example of a 9° N-7 DNA polymerase sequence is:

(SEQ ID NO: 3)
MILDTDYITENGKPVIRVFKKENGEFKIEYDRTFEPYFYALLKDDSAIE
DVKKVTAKRHGTVVKVKRAEKVQKKFLGRPIEVWKLYFNHPQDVPAIRD
RIRAHPAVVDIYEYDIPFAKRYLIDKGLIPMEGDEELTMLAFDIETLYH
EGEEFGTGPILMISYADGSEARVITWKKIDLPYVDVVSTEKEMIKRFLR
VVREKDPDVLITYNGDNFDFAYLKKRCEELGIKFTLGRDGSEPKIQRMG
DRFAVEVKGRIHFDLYPVIRRTINLPTYTLEAVYEAVFGKPKEKVYAEE
IAQAWESGEGLERVARYSMEDAKVTYELGREFFPMEAQLSRLIGQSLWD
VSRSSTGNLVEWFLLRKAYKRNELAPNKPDERELARRRGGYAGGYVKEP
ERGLWDNIVYLDFRSLYPSIIITHNVSPDTLNREGCKEYDVAPEVGHKF
CKDFPGFIPSLLGDLLEERQKIKRKMKATVDPLEKKLLDYRQRAIKILA
NSFYGYYGYAKARWYCKECAESVTAWGREYIEMVIRELEEKFGFKVLYA
DTDGLHATIPGADAETVKKKAKEFLKYINPKLPGLLELEYEGFYVRGFF
VTKKKYAVIDEEGKITTRGLEIVRRDWSEIAKETQARVLEAILKHGDVE
EAVRIVKEVTEKLSKYEVPPEKLVIHEQITRDLRDYKATGPHVAVAKRL
AARGVKIRPGTVISYIVLKGSGRIGDRAIPADEFDPTKHRYDAEYYIEN
QVLPAVERILKAFGYRKEDLRYQKTKQVGLGAWLKVKGKK.

In some embodiments, the DNA polymerase is DNA polymerase I or a functional fragment thereof (e.g., a Klenow fragment). Klenow fragment is an exonuclease deficient fragment of DNA polymerase I. An example of DNA polymerase I sequence is: MVQIPQNPLILVDGSSYLYRAYHAFPPLTNSAGEPTGAMYGVLNMLRSLIMQYKPTHAAV VFDAKGKTFRDELFEHYKSHRPPMPDDLRAQIEPLHAMVKAMGLPLLAVSGVEADDVIGT LAREAEKAGRPVLISTGDKDMAQLVTPNITLINTMTNTILGPEEVVNKYGVPPELIIDFLALM GDSSDNIPGVPGVGEKTAQALLQGLGGLDTLYAEPEKIAGLSFRGAKTMAAKLEQNKEVA YLSYQLATIKTDVELELTCEQLEVQQPAAEELLGLFKKYEFKRWTADVEAGKWLQAKGAK PAAKPQETSVADEAPEVTATVISYDNYVTILDEETLKAWIAKLEKAPVFAFDTETDSLDNIS ANLVGLSFAIEPGVAAYIPVAHDYLDAPDQISRERALELLKPLLEDEKALKVGQNLKYDRGI LANYGIELRGIAFDTMLESYILNSVAGRHDMDSLAERWLKHKTITFEEIAGKGKNQLTFNQI ALEEAGRYAAEDADVTLQLHLKMWPDLQKHKGPLNVFENIEMPLVPVLSRIERNGVKIDP KVLHNHSEELTLRLAELEKKAHEIAGEEFNLSSTKQLQTILFEKQGIKPLKKTPGGAPSTSEE VLEELALDYPLPKVILEYRGLAKLKSTYTDKLPLMINPKTGRVHTSYHQAVTATGRLSSTDP NLQNIPVRNEEGRRIRQAFIAPEDYVIVSADYSQIELRIMAHLSRDKGLLTAFAEGKDIHRAT AAEVFGLPLETVTSEQRRSAKAINFGLIYGMSAFGLARQLNIPRKEAQKYMDLYFERYPGV LEYMERTRAQAKEQGYVETLDGRRLYLPDIKSSNGARRAAAERAAINAPMQGTAADIIKR AMIAVDAWLQAEQPRVRMIMQVHDELVFEVHKDDVDAVAKQIHQLMENCTRLDVPLLVE VGSGENWDQAH (SEQ ID NO: 4). In some embodiments, a Klenow fragment includes positions 324-928 with respect to SEQ ID NO: 4.

In some embodiments, the polymerase is a reverse transcriptase. Reverse transcriptases typically have RNA-dependent DNA polymerase activity and DNA-dependent DNA polymerase activity. Examples of reverse transcriptases include Moloney murine leukemia virus (MMLV) reverse transcriptase, HIV-1 reverse transcriptase, and avian myeloblastosis virus (AMV) reverse transcriptase. In some embodiments, the reverse transcriptase lacks (e.g., is mutated to lack) ribonuclease activity. In some embodiments, ribonuclease activity degrade template particularly during longer incubation times such as when reverse transcribing longer cDNAs. In some embodiments, the polymerase is a reverse transcriptase and the template nucleic acid molecule is an RNA molecule. In some embodiments, the polymerase is a reverse transcriptase and the nucleotide molecules include deoxyribonucleotide molecules.

In some embodiments, the reverse transcriptase is an MMLV reverse transcriptase or a functional variant thereof. An example of an MMLV reverse transcriptase sequence is: AFPLERPDWDYTTQAGRNHLVHYRQLLLAGLQNAGRSPTNLAKVKGITQGPNESPSAFLER LKEAYRRYTPYDPEDPGQETNVSMSFIWQSAPDIGRKLGRLEDLKSKTLGDLVREAEKIFN KRETPEEREERIRRETEEKEERRRTVDEQKEKERDRRRHREMSKLLATVVIGQEQDRQEGE RKRPQLDKDQCAYCKEKGHWAKDCPKKPRGPRGPRPQTSLLTLGDXGGQGQDPPPEPRIT LKVGGQPVTFLVDTGAQHSVLTQNPGPLSDKSAWVQGATGGKRYRWTTDRKVHLATGK VTHSFLHVPDCPYPLLGRDLLTKLKAQIHFEGSGAQVVGPMGQPLQVLTLNIEDEYRLHETS KEPDVSLGFTWLSDFPQAWAESGGMGLAVRQAPLIIPLKATSTPVSIKQYPMSQEARLGIKP HIQRLLDQGILVPCQSPWNTPLLPVKKPGTNDYRPVQDLREVNKRVEDIHPTVPNPYNLLSG LPPSHQWYTVLDLKDAFFCLRLHPTSQPLFAFEWRDPEMGISGQLTWTRLPQGFKNSPTLF DEALHRDLADFR (SEQ ID NO: 5). Residues 431-560 of SEQ ID NO: 5 provide reverse transcriptase activity.

In some embodiments, the reverse transcriptase is an HIV-1 reverse transcriptase or a functional variant thereof. An example of an HIV-1 reverse transcriptase sequence is:

(SEQ ID NO: 6)
PISPIEPVPVKLKPGMDGPKVKQWPLTEEKIKALVEICTEMEKEGKISK
IGPENPYNTPVFAIKKKDSTRWRKLVDFRELNKRTQDFWEVQLGIPHPA
GLKKKRSVTVLDVGDAYFSVPLDKEFRKYTAFTIPSINNETPGIRYQYN
VLPQGWKGSPAIFQSSMTKILEPFRKQNPDIVIYQYMDDLYVGSDLEIG
QHRTKIEELRQHLLKWGFTTPDKKHQKEPPFLWMGYEHHPDKWTVQPIV
LPEKDSWTVNDIQK.

In some embodiments, the polymerase is selected from Taq polymerase, 9° N-7 DNA polymerase or a functional variant thereof (e.g., D141A/E143A/A485L), and a Klenow fragment of DNA polymerase I. In some embodiments, the polymerase is not labeled with a detectable label.

In some embodiments, the nucleotide in a set of unlabeled nucleotides is selected from A, T, U, C, and G. In some embodiments, the nucleotide in a set of unlabeled nucleotides is selected from A, T, C, and G.

Methods for processing the series of optical signals detected over the course of performing a cyclic series of base-by-base sequencing reactions to identify a nucleotide sequence are described elsewhere herein.

In some embodiments, a nucleotide is any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some embodiments, the nucleotides are terminated (e.g., reversibly terminated). In some embodiments, the nucleotides are conjugated to a detectable label, e.g., a fluorophore. In some embodiments, the nucleotides are conjugated to other moieties, e.g., reactive functional groups.

In some embodiments, the nucleotide comprises any suitable reversible terminator moiety. In some embodiments, the nucleotide is a 3′-O-blocked reversibly terminated nucleotide. In some embodiments, the 3′-O-blocked reversibly terminated nucleotide is, e.g., a 3′-O-azidomethyl deoxynucleotide triphosphate (3′-O-azidomethyl dNTP), a 3′-O-allyl deoxynucleotide triphosphate (3′-O-allyl-dNTP), 3′-O-acetate deoxynucleotide triphosphate (3′-O-acetate dNTP), or a 3′-O-amino deoxynucleotide triphosphate (3′-O—NH₂ dNTP). In some embodiments, the 3′ reversibly terminated nucleotide may be a 3′-unblocked reversibly terminated nucleotide.

### III. Kits and Systems

In some embodiments, provided herein are kits for sequencing nucleic acid molecules, including kits for sequencing and analysis of target nucleic acids in a biological sample according to any of the methods described herein.

In some embodiments, provided herein is a kit comprising any of the control sequencing primers, control probes or probe sets, and optionally additional probes or probe sets and second sequencing primers described herein. In some embodiments, the kit further comprises additional reagents such as primers for performing rolling circle amplification according to the methods described herein. In some embodiments, the kit further comprises any of the polymerases described herein.

In some aspects, provided herein is a kit for performing nucleic acid sequencing with error correction, comprising: (i) a control sequencing primer labeled with a first fluorescent dye; (ii) a control probe or probe set, wherein the control sequencing primer is capable of binding to a control sequence comprising a known sequence in the control probe or probe set or a product thereof; (iii) a second sequencing primer; (iv) a second probe or probe set, wherein the second probe or probe set is capable of binding to a target of interest, and wherein the second sequencing primer is capable of binding to a sequence of interest in the second probe or probe set or a product thereof; and (v) one or more reagents for performing a nucleic acid sequencing reaction. In some embodiments, the first fluorescent dye is a ultraviolet (UV), deep ultraviolet (DUV) or near ultraviolet (NUV) dye. In some embodiments, the first fluorescent dye is a NUV dye. In some embodiments, the first fluorescent dye is attached to the 5′ end of the control primer. In some embodiments, the first fluorescent dye is attached to the 5′ end of the control primer via a linker.

In some embodiments, the control probe or probe set binds to a housekeeping gene transcript in the biological sample. In some embodiments, the known sequence comprises at least four nucleotides, wherein at least one position of the known sequence is adenine (A), at least one position of the known sequence is thymine (T) or uracil (U), at least one position of the known sequence is guanine (G), and at least one position of the known sequence is cytosine (C). In some embodiments, at least one position of the known sequence is adenine (A), at least one position of the known sequence is thymine (T), at least one position of the known sequence is guanine (G), and at least one position of the known sequence is cytosine (C). In some embodiments, the known sequence comprises a repeating pattern of four nucleobase types. In some embodiments, the known sequence comprises a repeating pattern of four nucleobase types and the known sequence is 12 to 400 nucleotides in length (e.g., between 20 and 50 nucleotides in length, between 15 and 50 nucleotides in length, between 25 and 50 nucleotides in length, or between 25 and 60 nucleotides in length).

In some embodiments, the kit comprises one or more further components for performing the in situ sequencing reaction. In some embodiments, the one or more further components include a polymerase, a primer, a support for a tissue or cell sample, or any combination thereof. In some embodiments, the kit further comprises any of the circular probes and/or circularizable probes or probe sets disclosed herein. In some embodiments, the kit comprises a polymerase for rolling circle amplification.

In some embodiments, the kit further comprises one or more further components for performing the flow cell sequencing reaction. In some embodiments, the one or more further components include a polymerase, a primer, a flow cell, primers, adapters for sequencing library preparation, or any combination thereof.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some embodiments, the kit also comprises any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers.

In some embodiments, provided herein are systems for sequencing nucleic acid molecules in a biological sample, including the biological sample including, at a first location, amplified copies of a control sequence including a known sequence, a control sequencing primer attached to a first fluorescent dye, a sequencing primer specific for a sequence of interest, fluorescently labelled dNTPs, and a polymerase. The systems are useful for performing the sequencing methods as disclosed herein.

### IV. Biological Samples & Sample Preparation

A sample disclosed herein can be or derived from any biological sample. The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample comprises cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some instances, the biological sample may be provided on a substrate. In some instances, a substrate herein can be any support that is insoluble in aqueous liquid and which allows for positioning of biological samples, analytes, features, and/or reagents (e.g., probes) on the support. In some instances, a biological sample can be attached to a substrate. Attachment of the biological sample can be irreversible or reversible, depending upon the nature of the sample and subsequent steps in the analytical method. In certain instances, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some instances, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### Preparation

In some embodiments, the biological sample is harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, in some embodiments, grown samples, and samples obtained via biopsy or sectioning, are prepared as thin tissue sections using a mechanical cutting apparatus such as a vibrating blade microtome. As another alternative, in some embodiments, a thin tissue section is prepared by applying a touch imprint of a biological sample to a suitable substrate material.

In some embodiments, the thickness of the tissue section is a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, in some embodiments cryostat sections are used. In some embodiments, the cryostat sections are 10-20 μm thick. More generally, the thickness of a tissue section typically depends on the method used to prepare the section and the physical characteristics of the tissue, and therefore sections having a wide variety of different thicknesses can be prepared and used. For example, in some embodiments the thickness of the tissue section is at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, in some embodiments, multiple tissue sections are obtained from a surgical biopsy sample by performing serial sectioning of the biopsy sample using a sectioning blade. In some embodiments, spatial information among the serial sections is preserved in this manner, and the sections are analysed successively to obtain three-dimensional information about the biological sample.

In some instances, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some instances, the biological sample is prepared using formalin-fixation and paraffin-embedding (FFPE), which are established methods. In some instances, cell suspensions and other non-tissue samples are prepared using formalin-fixation and paraffin-embedding. Following fixation of the sample and embedding in a paraffin or resin block, in some embodiments the sample is sectioned as described above. Prior to analysis, in some embodiments the paraffin-embedding material removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, in some embodiments a biological sample is fixed in any of a variety of other fixatives to preserve the biological structure of the sample prior to analysis. For example, in some embodiments a sample is fixed via immersion in ethanol, methanol, acetone, paraformaldehyde (PFA)-Triton, and combinations thereof.

In some instances, the methods provided herein comprise one or more post-fixing (also referred to as post-fixation) steps. In some instances, one or more post-fixing step is performed after contacting a sample with a polynucleotide disclosed herein, e.g., one or more probes such as a circular or circularizable probe (e.g., padlock probe). In some instances, one or more post-fixing step is performed after a hybridization complex comprising a probe and a target is formed in a sample. In some instances, one or more post-fixing step is performed prior to a ligation reaction disclosed herein.

In some instances, a method disclosed herein comprises de-crosslinking the reversibly cross-linked biological sample. The de-crosslinking does not need to be complete. In some instances, only a portion of crosslinked molecules in the reversibly cross-linked biological sample are de-crosslinked and allowed to migrate.

In some instances, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some instances, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some instances, the biological sample is permeabilized by any suitable methods. For example, in some embodiments one or more lysis reagents are added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, in some embodiments a surfactant-based lysis solution is used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some instances, DNase and Rnase inactivating agents or inhibitors such as proteinase K, and/or chelating agents such as EDTA, can be added to the sample. For example, a method disclosed herein comprises a step for increasing accessibility of a nucleic acid for binding, e.g., a denaturation step to open up DNA in a cell for hybridization by a probe. For example, proteinase K treatment may be used to free up DNA with proteins bound thereto.

### Embedding

In some instances, the biological sample is embedded in a matrix (e.g., a hydrogel matrix). Embedding the sample in this manner typically involves contacting the biological sample with a hydrogel such that the biological sample becomes surrounded by the hydrogel. For example, in some embodiments, the sample is embedded by contacting the sample with a suitable polymer material, and activating the polymer material to form a hydrogel. In some instances, the hydrogel is formed such that the hydrogel is internalized within the biological sample. Biological samples can include analytes (e.g., protein, RNA, and/or DNA) embedded in a 3D matrix. In some instances, amplicons (e.g., rolling circle amplification products) derived from or associated with analytes (e.g., protein, RNA, and/or DNA) can be embedded in a 3D matrix. In some instances, a 3D matrix comprises a network of natural molecules and/or synthetic molecules that are chemically and/or enzymatically linked, e.g., by crosslinking. In some instances, a 3D matrix comprises a synthetic polymer. In some instances, a 3D matrix comprises a hydrogel.

In some embodiments, a biological sample is embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some instances, the biological sample is embedded in a matrix (e.g., a hydrogel matrix). Embedding the sample in this manner typically involves contacting the biological sample with a hydrogel such that the biological sample becomes surrounded by the hydrogel. For example, in some instances the sample embedded by contacting the sample with a suitable polymer material, and activating the polymer material to form a hydrogel. In some instances, the hydrogel is formed such that the hydrogel is internalized within the biological sample.

In some instances, the biological sample is immobilized in the hydrogel via cross-linking of the polymer material that forms the hydrogel. In some embodiments, cross-linking is performed chemically and/or photochemically, or alternatively by any other suitable hydrogel-formation method.

In some instances, the biological sample is reversibly cross-linked prior to or during an in situ assay. In some embodiments, the analytes, polynucleotides and/or amplification product (e.g., amplicon) of an analyte or a probe bound thereto is anchored to a polymer matrix. For example, the polymer matrix can be a hydrogel. In some instances, one or more of the polynucleotide probe(s) and/or amplification product (e.g., amplicon) thereof are modified to contain functional groups that can be used as an anchoring site to attach the polynucleotide probes and/or amplification product to a polymer matrix. In some instances, a modified probe comprising oligo dT may be used to bind to mRNA molecules of interest, followed by reversible or irreversible crosslinking of the mRNA molecules.

In some instances, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some instances, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some instances, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, in some embodiments a hydrogel-matrix is formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

In some instances, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some instances, hydrogel formation occurs after contacting one or more second materials during formation of the substrate. Hydrogel formation can also occur within a structure (e.g., wells, ridges, projections, and/or markings) located on a substrate.

In some instances, hydrogel formation on a substrate occurs before, contemporaneously with, or after probes are provided to the sample. For example, hydrogel formation can be performed on the substrate already containing the probes.

In some instances, hydrogel formation occurs within a biological sample. In some embodiments, a biological sample (e.g., tissue section) is embedded in a hydrogel. In some instances, hydrogel subunits are infused into the biological sample, and polymerization of the hydrogel is initiated by an external or internal stimulus.

In instances in which a hydrogel is formed within a biological sample, functionalization chemistry can be used. In some instances, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some instances, hydrogel formation within a biological sample is permanent. For example, biological macromolecules can permanently adhere to the hydrogel allowing multiple rounds of interrogation. In some instances, hydrogel formation within a biological sample is reversible. In some instances, HTC reagents are added to the hydrogel before, contemporaneously with, and/or after polymerization. In some instances, a cell labeling agent is added to the hydrogel before, contemporaneously with, and/or after polymerization. In some instances, a cell-penetrating agent is added to the hydrogel before, contemporaneously with, and/or after polymerization.

In some instances, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some instances, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some instances, a hydrogel-embedded sample is stored before or after clearing of hydrogel, in a medium (e.g., a mounting medium, methylcellulose, or other semi-solid mediums).

In some instances, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some instances, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some instances, the sample is isometrically expanded to at least 2× and less than 20× of its non-expanded size.

### Staining and Immunohistochemistry (IHC)

To facilitate visualization, in some embodiments biological samples are stained using a wide variety of stains and staining techniques. In some instances, for example, a sample is stained using any number of stains and/or immunohistochemical reagents. One or more staining steps may be performed to prepare or process a biological sample for an assay described herein or may be performed during and/or after an assay. In some instances, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some instances, cells in the sample are segmented using one or more images taken of the stained sample.

In some instances, the stain is performed using a lipophilic dye. In some examples, the staining is performed with a lipophilic carbocyanine or aminostyryl dye, or analogs thereof (e.g., DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some instances, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some instances, the sample can be stained using Romanowsky stain, including Wright's stain, Jenner's stain, Can-Grunwald stain, Leishman stain, and Giemsa stain.

In some instances, biological samples are destained. Any suitable methods of destaining or discoloring a biological sample may be utilized and generally depend on the nature of the stain(s) applied to the sample. For example, in some instances, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### V. Analytes

A biological sample may comprise one or a plurality of analytes of interest. Methods for performing multiplexed assays to analyze two or more different analytes in a single biological sample are provided.

The methods and compositions disclosed herein can be used to detect and analyze a wide variety of different analytes. In some embodiments, an analyte can include any biological substance, structure, moiety, or component to be analyzed. In some embodiments, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

### Labeling Agents

In some instances, provided herein are methods and compositions for analyzing endogenous analytes (e.g., RNA, ssDNA, cell surface or intracellular proteins, and/or metabolites) in a sample using one or more labeling agents. In some instances, an analyte labeling agent may include an agent that interacts with an analyte (e.g., an endogenous analyte in a sample). In some instances, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some instances, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some instances, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some instances, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some instances, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of non-limiting examples of labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some instances, an analyte binding moiety includes one or more antibodies or epitope-binding fragments thereof. The antibodies or epitope-binding fragments including the analyte binding moiety can specifically bind to a target analyte. In some instances, the analyte is a protein (e.g., a protein on a surface of the biological sample (e.g., a cell) or an intracellular protein). In some instances, a plurality of analyte labeling agents comprising a plurality of analyte binding moieties bind a plurality of analytes present in a biological sample. In some instances, the plurality of analytes includes a single species of analyte (e.g., a single species of polypeptide). In some instances in which the plurality of analytes includes a single species of analyte, the analyte binding moieties of the plurality of analyte labeling agents are the same. In some instances in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some instances, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some embodiments, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some instances, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some instances, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

### Detectable Labels

In some instances, one or more nucleotides (or analogs thereof), primers, and optionally probes can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding of the nucleotide is sufficiently maintained to enable identification of the complementary base on the template nucleic acid correctly. In some instances, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some instances, the detectable label is bound to another moiety, for example, a nucleotide or nucleotide analog, and can include a fluorescent, a colorimetric, or a chemiluminescent label.

In some instances, a detectable label can be attached to another moiety, for example, a nucleotide or nucleotide analog. In some instances, one or more nucleotides can be labeled with a cleavable detectable tag or label. For example, the non-terminating fluorescently labeled nucleotides can include a DBCO-nucleotide conjugated to fluorescent compound with a disulfide linker. In some instances, a non-terminating fluorescently labeled nucleotide is incorporated into the strand without termination, and after imaging, the linker can be cleaved to remove fluorescent label. In some instances, a DBCO-nucleotide (e.g., 5-DBCO-PEG₄-UTP) can undergo a click reaction with the cleavable linker conjugated to a fluorescent label (e.g., cleavable linker-fluorophore), and a disulfide group can be cleaved by tris(2-carboxyethyl)phosphine (TCEP) reduction together with 3′-O-azidomethyl-dNTP.

In some embodiments, the detectable label is a fluorophore. Examples of fluorophores include but are not limited to phycoerythrin, ALEXA FLUOR™ dyes (fluorescent dye), fluorescein, YPet, CyPet, Cascade® blue (fluorescent dye), allophycocyanin, Cy3™ (cyanine 3), Cy5™ (cyanine 5), Cy7™ (cyanine 7), and Texas Red® (fluorescent dye).

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

### Generation of Products

In some instances, provided herein are methods and compositions for analyzing one or more products of an endogenous analyte and/or a labeling agent in a biological sample. In some instances, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some instances, a labeling agent that directly or indirectly binds to an analyte in the biological sample is analyzed. In some instances, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some instances, a hybridization product comprising the pairing of substantially complementary or complementary nucleic acid sequences within two different molecules can be analyzed. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Non-limiting examples of barcoded probes or probe sets may be based on a circularizable probe (e.g., a padlock probe), a gapped circularizable probe (e.g., a gapped padlock probe), a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some instances, a ligation product of an endogenous analyte and/or a labeling agent can be analyzed. In some instances, the ligation product is formed between two or more endogenous analytes. In some instances, the ligation product is formed between two or more labeling agents. In some instances, the ligation product is an intramolecular ligation of an endogenous analyte. In some instances, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some instances, provided herein is a probe or probe set capable of DNA-templated ligation, such as from a cDNA molecule. See, e.g., U.S. Pat. No. 8,551,710, which is hereby incorporated by reference in its entirety. In some instances, provided herein is a probe or probe set capable of RNA-templated ligation. See, e.g., U.S. Pat. Pub. 2020/0224244 which is hereby incorporated by reference in its entirety. In some instances, the probe set is a SNAIL probe set. See, e.g., U.S. Pat. Pub. 20190055594, which is hereby incorporated by reference in its entirety. In some instances, provided herein is a multiplexed proximity ligation assay. See, e.g., U.S. Pat. Pub. 20140194311 which is hereby incorporated by reference in its entirety. In some instances, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some instances, a circular probe can be indirectly hybridized to the target nucleic acid. In some instances, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some instances, the ligation involves chemical ligation (e.g., click chemistry ligation). In some instances, the chemical ligation involves template dependent ligation. In some instances, the chemical ligation involves template independent ligation. In some instances, the click reaction is a template-independent reaction (see, e.g., Xiong and Seela (2011), J. Org. Chem. 76(14): 5584-5597, incorporated by reference herein in its entirety). In some instances, the click reaction is a template-dependent reaction or template-directed reaction. In some instances, the template-dependent reaction is sensitive to base pair mismatches such that reaction rate is significantly higher for matched versus unmatched templates. In some instances, the click reaction is a nucleophilic addition template-dependent reaction. In some instances, the click reaction is a cyclopropane-tetrazine template-dependent reaction.

In some instances, the ligation involves enzymatic ligation. In some instances, the enzymatic ligation involves use of a ligase. In some embodiments, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some instances, the ligase is a T4 RNA ligase. In some instances, the ligase is a splintR ligase. In some instances, the ligase is a single stranded DNA ligase. In some instances, the ligase is a T4 DNA ligase. In some instances, the ligase is a ligase that has an DNA-splinted DNA ligase activity. In some instances, the ligase is a ligase that has an RNA-splinted DNA ligase activity.

In some instances, the ligation herein is a direct ligation. In some instances, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some instances, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific instances, the gap may be a gap of about 1, 2, 3, 4, 5, 6, 7, 8, 9 or 10 or more nucleotides, of any integer (or range of integers) of nucleotides in between the indicated values. In some instances, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some instances, the ligation herein is preceded by gap filling. In other instances, the ligation herein does not require gap filling.

In some instances, ligation of the polynucleotides produces polynucleotides with melting temperature higher than that of unligated polynucleotides. Thus, in some embodiments, ligation stabilizes the hybridization complex containing the ligated polynucleotides prior to subsequent steps, comprising amplification and detection.

In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some instances, the ligation herein is a proximity ligation of ligating two (or more) nucleic acid sequences that are in proximity with each other, e.g., through enzymatic means (e.g., a ligase). In some instances, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some instances, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some instances, a product of an endogenous analyte and/or a labeling agent is an amplification product of one or more polynucleotides, for instance, a circular probe or circularizable probe or probe set. In some instances, the amplifying is achieved by performing rolling circle amplification (RCA). In other instances, a primer that hybridizes to the circular probe or circularized probe is added and used as such for amplification. In some instances, the RCA comprises a linear RCA, a branched RCA, a dendritic RCA, or any combination thereof.

In some instances, the amplification is performed at a temperature between or between about 20° C. and about 60° C. In some instances, the amplification is performed at a temperature between or between about 30° C. and about 40° C. In some embodiments, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some instances, upon addition of a DNA polymerase in the presence of appropriate dNTP precursors and other cofactors, a primer is elongated to produce multiple copies of the circular template. This amplification step can utilize isothermal amplification or non-isothermal amplification. In some instances, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801, all of which are hereby incorporated by reference herein in their entireties). Non-limiting examples of polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some embodiments, DNA polymerases that have been engineered or mutated to have desirable characteristics can be employed. In some instances, the polymerase is phi29 DNA polymerase.

In some embodiments, during the amplification step, modified nucleotides can be added to the reaction to incorporate the modified nucleotides in the amplification product (e.g., nanoball). Non-limiting examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some embodiments, the amplification products comprises a modified nucleotide, such as an amine-modified nucleotide. In some instances, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the polynucleotides and/or amplification product (e.g., amplicon) can be anchored to a polymer matrix. For example, the polymer matrix can be a hydrogel. In some instances, one or more of the polynucleotide probe(s) can be modified to contain functional groups that can be used as an anchoring site to attach the polynucleotide probes and/or amplification product to a polymer matrix. Non-limiting examples of modification and polymer matrix that can be employed in accordance with the provided instances comprise those described in, for example, WO 2014/163886, WO 2017/079406, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some embodiments, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some instances, the provided methods involve embedding the one or more polynucleotide probe sets and/or the amplification products in the presence of hydrogel subunits to form one or more hydrogel-embedded amplification products. In some instances, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some instances, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some instances, the RCA template may comprise the target analyte, or a part thereof, where the target analyte is a nucleic acid, or it may be provided or generated as a proxy, or a marker, for the analyte. In some instances, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some instances, a product herein includes a molecule or a complex generated in a series of reactions, e.g., hybridization, ligation, extension, replication, transcription/reverse transcription, and/or amplification (e.g., rolling circle amplification), in any suitable combination.

### VI. Fluorescence Detection and Imaging

### Fluorescence Detection

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some instances, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of fluorescent labels and nucleotides and/or polynucleotides conjugated to such fluorescent labels comprise those described elsewhere herein and those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), all of which are hereby incorporated by reference herein in their entireties. In some instances, non-limiting examples of techniques and methods applicable to the provided embodiments comprise those described in, for example, U.S. Pat. Nos. 4,757,141, 5,151,507 and 5,091,519, all of which are hereby incorporated by reference herein in their entireties. In some instances, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are hereby incorporated by reference herein in their entireties. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are hereby incorporated by reference herein in their entireties. In some instances, a fluorescent label comprises a signaling moiety that conveys information through the fluorescence absorption and/or emission properties of one or more molecules. Non-limiting examples of fluorescence properties comprise fluorescence intensity, fluorescence lifetime, emission spectrum characteristics and energy transfer.

### Imaging

In some embodiments, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some instances, fluorescence microscopy is used for detection and imaging of the sample. In some embodiments, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be or comprise any microscope that uses fluorescence to generate an image, whether it is a more simple set up like an epifluorescence microscope, or a more complicated design such as a confocal microscope, which uses optical sectioning to achieve better z-axis resolution of the sample to be imaged.

In some instances, confocal microscopy is used for detection and imaging of the sample. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immune-stained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some instances, a method herein comprises subjecting the sample to expansion microscopy methods and techniques. Expansion allows individual targets (e.g., mRNA or RNA transcripts) which are densely packed within a cell, to be resolved spatially in a high-throughput manner. Expansion microscopy techniques can be performed, such as those described in US 2016/0116384 and Chen et al., Science, 347, 543 (2015), each of which are incorporated herein by reference in their entirety. In some instances, the method does not comprise subjecting the sample to expansion microscopy. In some instances, the method does not comprise dissociating a cell from the sample such as a tissue or the cellular microenvironment. In some instances, the method does not comprise lysing the sample or cells therein. In some instances, the method does not comprise embedding the sample or molecules from the sample in an exogenous matrix.

In some cases, analysis is performed on one or more images captured, and may comprise processing the image(s) and/or quantifying signals observed. In some instances, images of signals from different fluorescent channels and/or nucleotide incorporation cycles can be compared and analyzed. In some instances, images of signals (or absence thereof) at a particular location in a sample from different fluorescent channels and/or sequential incorporation cycles can be aligned to analyze an analyte at the location. For instance, a particular location in a sample can be tracked and signal spots from sequential incorporation cycles can be analyzed to detect a target polynucleotide sequence (e.g., a barcode sequence or subsequence thereof) in an analyte at the location. The analysis may comprise processing information of one or more cell types, one or more types of analytes, a number or level of analyte, and/or a number or level of cells detected in a particular region of the sample. In some instances, the analysis comprises detecting a sequence e.g., a barcode sequence present in an amplification product at a location in the sample. In some instances, the number of signals detected in a unit area in the biological sample is quantified. In some instances, the signals detected at a corresponding position in the biological sample in a plurality of images taken at different z positions (e.g., in the depth direction) is quantified and analyzed.

### VI. Barcode Sequences

In some instances, an analyte described herein can be associated with one or more barcode(s), e.g., at least two, three, four, five, six, seven, eight, nine, ten, or more barcodes (e.g., barcodes comprised by sequences of interest that are sequenced according to the provided methods). Barcodes can be used to spatially-resolve molecular components found in biological samples, for example, within a cell or a tissue sample. A barcode can be attached to an analyte or to another moiety or structure (e.g., a target-specific antibody) in a reversible or irreversible manner. In some embodiments, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, a barcode includes two or more sub-barcodes (or barcode segments) that together function as a single barcode. For example, a polynucleotide barcode can include two or more polynucleotide sequences (e.g., sub-barcodes) that are contiguous or that are separated by one or more non-barcode sequences. In some instances, a barcode may comprise about 1, 2, 3, 4, 5, 6, 7, 8, 9, 10, or more than 10 sub-barcodes (or barcode segments). In some instances, each sub-barcode (or barcode segment) may comprise about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some instances, each non-barcode sequence may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some instances, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some instances, e.g., in a barcode sequencing method, barcode sequences are detected for identification of other molecules including nucleic acid molecules (DNA or RNA) that are longer than the barcode sequences themselves, as opposed to direct sequencing of the longer nucleic acid molecules. In some instances, an N-mer barcode sequence can comprise up to 4^{N} unique sequences given a sequencing read of N bases, and a much shorter sequencing read may be required for molecular identification compared to non-barcoded sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some instances, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

### VII. Cyclic Array Sequencing in a Flow Cell

In some instances, a method of sequencing as provided herein includes “cyclic array sequencing” of amplified template nucleic acid molecules. Cyclic array flow cell sequencing methods generally involve performing multiple cycles of an enzymatic reaction on an array of spatially-separated oligonucleotide features (e.g., clonally-amplified colonies of template nucleic acid fragments tethered to a support surface, e.g., a flow cell surface). In some instances, the template nucleic acid is modified with known adapter sequence(s) comprising, e.g., amplification and/or sequencing primer binding sites, and then affixed to the support surface (e.g., the lumen surface(s) of a flow cell) in a random or patterned array by hybridization to surface-tethered complementary capture probes (complementary to adapter sequences) on the support surface, clonally amplified, and then probed using the aforementioned sequencing reaction as described herein. In some embodiments, the flow cell sequencing comprises massively parallel sequencing reaction, whereby each enzymatic reaction cycle is used to query only one base (the “interrogation” nucleobase) of the template nucleic acid fragment in each oligonucleotide feature, but thousands to billions of template nucleic acid molecules may be processed in parallel. Performing repeated cycles is then used to progressively identify the nucleic acid sequence of each template nucleic acid molecule based on patterns of detection of a signal or detection of an absence of a signal associated with binding of a nucleotide to the template, as detected over the course of multiple reaction cycles.

### Nucleic Acid Extraction

Nucleic acid extraction from cells or other biological samples may be performed using any of a variety of techniques. For example, a typical DNA extraction procedure may comprise: (i) collection of a cell or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant (e.g., using spin columns or paramagnetic beads) to remove detergents, proteins, salts, or other reagents used during the cell membrane lysis step. Non-limiting examples of methods for performing nucleic acid (e.g., DNA and RNA) extraction are described in, for example, Ali et al. (2017) “Current Nucleic Acid Extraction Methods and Their Implications to Point-of-Care Diagnostics”, BioMed Research International 2017:9306564, and Dairawan et al. (2020), “The Evolution of DNA Extraction Methods”, Am J Biomed Sci & Res 8(1):39-45, the entire contents of each of which are incorporated herein by reference.

A variety of suitable commercial nucleic acid extraction and purification kits are consistent with the disclosure herein. Examples include, but are not limited to, the QIAamp® kits (for isolation of genomic DNA from human samples) and DNAeasy kits (for isolation of genomic DNA from animal or plant samples) from Qiagen (Germantown, Md.), or the Maxwell® and ReliaPrep™ series of kits from Promega (Madison, Wis.).

### Sequencing Library Preparation

Sequence library preparation may be performed using any of a variety of techniques. Library preparation typically comprises performing the steps of, e.g., end repair, tailing, and ligation of adapter sequences to template nucleic acid fragments. Extracted nucleic acid molecules (e.g., DNA molecule), or fragments thereof, that are typically used as the input for sequencing library preparation often have overhangs containing single-stranded DNA (ssDNA overhangs), breaks in the phosphodiester backbone that exist on just one strand (nicks), and/or ssDNA regions internal to the duplex molecule (ssDNA gaps). End repair reactions (using, e.g., a combination of 3′ exonuclease digestion to remove 3′ overhangs and a strand displacing polymerase reaction using dNTPs to fill nicks and gaps) are used to correct these defects in order to maximize the yield for capturing and sequencing the extracted DNA, and result in the generation of blunt-ended, double-stranded DNA (dsDNA) molecules.

Tailing (e.g., A tailing) is an enzymatic method (using, e.g., a Taq DNA polymerase) for adding a non-templated nucleotide (e.g., an A nucleotide) to the 3′ end of a blunt-ended, double-stranded DNA molecule that facilitates the ligation of the adapter sequences used for sequencing.

One or more adapter sequences may then be ligated to the ends of the end-repaired and tailed template nucleic acid molecules. The adapter sequences may comprise, for example, (i) capture sequences (e.g., the Illumina p5 and p7 adapter sequences) that allow the nucleic acid molecules of the library to bind to a flow cell surface comprising complementary capture probes, (ii) amplification primer binding sites for use in performing reverse transcription and/or for generating clonally-amplified clusters on a flow cell surface, (iii) sequencing primer binding sites (e.g., the Illumina Rd1 and Rd2 sequencing primer binding site sequences) used to initiate sequencing. In addition to amplification and/or sequencing primer binding sites, in some instances the adapters may comprise a barcode sequence, e.g., a sample identification barcode sequence (such as the Illumina Index 1 and Index 2 sample identifier sequences).

Non-limiting examples of methods for performing sequencing library preparation are described in, for example, Head et al. (2014), “Library construction for next-generation sequencing: Overviews and challenges”, BioTechniques 56(1):61-77, and Hess et al. (2020), “Library preparation for next generation sequencing: A review of automation strategies”, Biotechnology Advances 41:107537, the entire contents of each of which are incorporated herein by reference.

### Nucleic Acid Amplification (Including Clonal Amplification)

In some instances, the disclosed methods for performing nucleic acid sequencing (e.g., in vitro and/or flow cell sequencing) may comprise performing one or more steps (e.g., 1, 2, 3, 4, 5, or more than 5) steps of nucleic acid amplification. Amplification reactions with respect to in situ based sequencing methods as described herein are discussed previously. In some instances, for example, one or more steps of nucleic acid amplification may be performed as part of sequencing library preparation and/or following sequencing library preparation. In some instances, one or more steps of nucleic acid amplification (e.g., using a solid-phase amplification technique such as bridge amplification) may be performed after the template molecules of a sequencing library have been tethered to a support surface (e.g., a flow cell surface) to generate clonally-amplified colonies of the tethered template nucleic acid fragments.

In some instances, nucleic acid amplification may be performed to amplify all of the nucleic acid molecules extracted from a biological sample (e.g., using a random primer sequence). In some instances, nucleic acid amplification may be performed to amplify a selected subset of nucleic acid molecules extracted from a biological sample (e.g., using one or more primer sequences designed to hybridize to portions of the sequences for one or more target nucleic acid molecules of interest, or to sequences adjacent thereto).

In some instances, nucleic acid amplification may be performed to amplify an entire sequencing library (e.g., using a primer sequence that hybridizes to a common amplification primer binding site in the sequencing library adapters). In some instances, nucleic acid amplification may be performed to amplify selected portions of the sequencing library (e.g., using one or more primer sequences designed to hybridize to one or more amplification primer binding sites associated with one or more identifier sequences (or barcodes) included in the sequencing library adapters).

Nucleic acid amplification may be performed using any of a variety of nucleic acid amplification techniques, including both thermal and/or isothermal nucleic acid amplification techniques. Examples of suitable thermal nucleic acid amplification techniques include, but are not limited to, polymerase chain reaction (PCR), multiplexed PCR, nested PCR, bridge PCR, reverse transcription PCR (RT-PCR). Examples of suitable isothermal nucleic acid amplification techniques include, but are not limited to, rolling circle amplification (RCA), nucleic acid sequence-based amplification (NASBA), loop-mediated isothermal amplification (LAMP), strand displacement amplification (SDA), helicase-dependent amplification (HDA), nicking enzyme amplification reaction (NEAR), and recombinase polymerase amplification (RPA). Examples of methods for performing nucleic acid amplification are described in, for example, Gill et al. (2008), “Nucleic Acid Isothermal Amplification Technologies—A Review”, Nucleosides, Nucleotides, and Nucleic Acids 27:224-243, Fakruddin et al. (2013), “Nucleic acid amplification: Alternative method of polymerase chain reaction”, J Pharm Bioallied Sci. 5(4): 245-252, and U.S. Pat. No. 8,143,008, the entire contents of each of which are incorporated herein by reference.

### Nucleotides

In some instances, the disclosed methods for performing nucleic acid sequencing (e.g., in situ and/or flow cell sequencing) may comprise the use of modified versions (e.g., comprising a first functional group or a second function group, as described elsewhere herein) of any of a variety of naturally-occurring nucleotides and/or functional analogs thereof (e.g., nucleotide analogs capable of hybridizing to a nucleic acid sequence in a sequence-specific/correctly base-paired manner) to probe a template nucleic acid sequence. Naturally-occurring nucleotides include deoxyribonucleotides (found in DNA) that comprise a deoxyribose sugar moiety, and ribonucleotides (found in RNA) that comprise a ribose sugar moiety. Naturally-occurring deoxyribonucleotides comprise a nucleobase (or “base”) selected from the group consisting of adenine (A), thymine (T), cytosine (C), or guanine (G). Naturally-occurring ribonucleotides comprise a nucleobase selected from the group consisting of uracil (U), adenine (A), cytosine (C), or guanine (G). In some instances, the nucleotides may be terminated (e.g., reversibly terminated). In some instances, the nucleotides may be conjugated to a detectable label, e.g., a fluorophore. In some instances, the nucleotides may be conjugated to other moieties, e.g., reactive functional groups.

### Primers (or Primer Sequences)

In some instances, the disclosed methods for performing nucleic acid sequencing (e.g., in situ and/or flow cell sequencing) can comprise the use of primer sequences that are complementary to, e.g., a subsequence (or primer binding site) that is part of a sequence of interest, such as a sequence of interest comprising an endogenous nucleic acid target sequence or a sequence (or primer binding site) that is located at or near a barcode (identifier) sequence associated with a target analyte. In some instances, a primer sequence may be designed to hybridize to a primer binding site associated with a single target analyte sequence and/or an associated target-specific barcode sequence. In some instances, a primer sequence may be designed to hybridize to a sequence (or primer binding site) that is associated with a plurality of target analyte sequences and/or associated target-specific barcode sequences (e.g., at least 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000, or more than 1000 target analyte sequences and/or associated target-specific barcode sequences).

### Polymerases

In some instances, the disclosed methods for performing nucleic acid sequencing (e.g., in situ and/or flow cell sequencing) may comprise performing one or more steps of nucleic acid amplification or replication using one or more polymerases. Examples of polymerases that may be used for amplification include, but are not limited to, DNA polymerases (e.g., Taq DNA polymerase), RNA polymerases, and/or reverse transcriptases.

As noted elsewhere herein, non-limiting examples of polymerases for use in rolling circle amplification (RCA) comprise DNA polymerases such phi29 (φ29) polymerase, Klenow fragment, Bacillus stearothermophilus DNA polymerase (BST), T4 DNA polymerase, T7 DNA polymerase, or DNA polymerase I. In some embodiments, DNA polymerases that have been engineered or mutated to have desirable characteristics can be employed. In some embodiments, the polymerase is phi29 DNA polymerase.

### VIII. Opto-Fluidic Instrument

In some instances, the sequencing methods described herein (e.g., in situ sequence or flow cell sequencing) include using instruments having integrated optics and fluidics modules (“opto-fluidic instruments” or “opto-fluidic systems”) for detecting target molecules (e.g., nucleic acids, proteins, antibodies, etc.) in biological samples (e.g., one or more cells or a tissue sample) as described herein.

In an opto-fluidic instrument, the fluidics module is configured to deliver one or more reagents (e.g., compositions comprising nucleotides, primers, polymerases and/or other enzymes, deprotection reagents, buffers, etc.) to the biological sample (e.g., to a sample cartridge within which the biological sample is contained) or to a flow cell (e.g., within which nucleic acid molecules extracted from the biological sample have been tethered) and/or to remove spent reagents therefrom. In some instances, one or more sample preparation steps (e.g., fixing, embedding, sample clearing, and/or nucleic acid extraction (in the case that nucleic acid molecules are to be extracted and sequenced in a flow cell)) may be performed prior to the sample being placed on the instrument. In some instances, the fluidics module is configured to deliver one or more further reagents (e.g., primary probe(s) such as circular probe(s) or circularizable probe(s) or probe set(s)) and/or to remove non-specifically hybridized probe(s). In some instances, the fluidics module is configured to deliver one or more detectably labeled probes and optionally intermediate probes to detect the target analytes, or amplified representatives thereof (e.g., RCP(s)) in the biological sample. In some instances, the fluidics module is configured to deliver one or more composition (e.g., compositions comprising nucleotides, primers, polymerases and/or other enzymes, deprotection reagents, buffers, etc.) to sequence, e.g., native nucleic acid sequences, barcode sequences associated with target analytes, or amplified copies thereof (e.g., barcode sequences included in RCP(s)) in the biological sample. In some instances, the fluidics module is configured to deliver one or more compositions (e.g., compositions comprising nucleotides, primers, polymerases and/or other enzymes, deprotection reagents, buffers, etc.) to a flow cell to sequence, e.g., native nucleic acid sequences, barcode sequences, or amplified copies thereof extracted from the biological sample.

Additionally, the optics module is configured to illuminate the biological sample (or flow cell) with light having one or more spectral emission curves (over a range of wavelengths) and subsequently capture one or more images of emitted light signals from the biological sample (or flow cell) during one or more decoding (e.g., probing or sequencing) cycles. In various instances, the captured images may be processed in real time and/or at a later time to determine the presence of the one or more target molecules in the biological sample, as well as two-dimensional and/or three-dimensional position information associated with each detected target molecule within the biological sample. In various instances, the captured images of a flow cell surface are processed in real time and/or at a later time to determine the sequence of the one or more nucleic acid sequences (e.g., barcode sequences associated with one or more target molecules) that have been extracted from a biological sample. In some embodiment, the optics module further comprises an autofocus mechanism configured to maintain focus at a specified sample plane (e.g., a plane that is perpendicular to the optical axis of an objective lens of the optics module).

Additionally, the opto-fluidics instrument includes a sample module configured to receive (and, optionally, secure) one or more biological samples (e.g., biological samples contained with one or more sample cartridges), or to receive (and, optionally, secure) one or more flow cells. In some instances, the sample module includes an X-Y stage configured to move the biological sample (or flow cell) along an X-Y plane (e.g., perpendicular to the optical axis of an objective lens of the optics module).

In various instances, the opto-fluidic instrument is configured to analyze one or more target molecules (e.g., one or more target RNAs) in their naturally occurring place (i.e., in situ) within the biological sample. In some instances, the opto-fluidic instrument is configured to analyze one or more target RNAs in relative spatial locations within the biological sample. For example, an opto-fluidic instrument may be an in-situ analysis system used to analyze a biological sample and detect target molecules including, but not limited to, DNA, RNA, proteins, antibodies, and/or the like. In some instances, the in situ analysis system is used to detect one or more target RNAs using target-primed rolling circle amplification (RCA) according to the methods disclosed herein.

In various instances, the opto-fluidic instrument may be configured to perform in situ target molecule detection via base-by-base sequencing (e.g., by sequencing an identifier sequence such as a barcode sequence associated with a target molecule) and/or any imaging or target molecule detection technique. That is, for example, an opto-fluidic instrument may include a fluidics module that includes fluids needed for establishing the experimental conditions required for the probing or sequencing of target molecules (or associate barcode sequences) in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

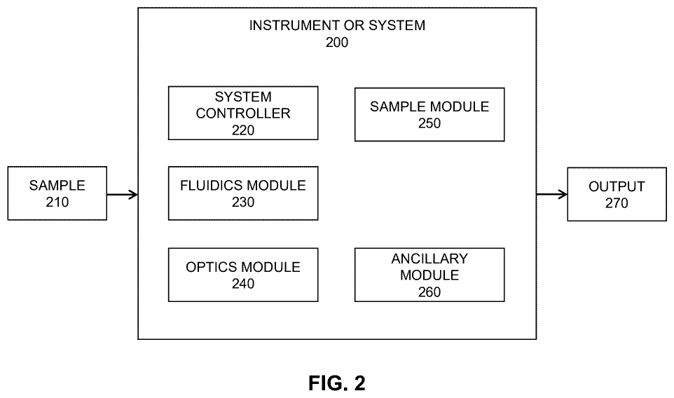

FIG. 2 shows an example workflow of analysis of a biological sample 210 (e.g., cell or tissue sample) using an opto-fluidic instrument or system 200, according to various instances. In various instances, the sample 210 can be a biological sample (e.g., a tissue) that includes molecules such as DNA, RNA, proteins, antibodies, etc. For example, the sample 210 can be a sectioned tissue that is treated to access the RNA thereof for probe (e.g., circularizable probe) hybridization and sequencing (e.g., using a sequencing primer that hybridizes to RCPs to sequence barcode sequences in the RCPs) described elsewhere herein.

In various instances, the sample 210 may be placed in the opto-fluidic instrument or system 200 for analysis and detection of the molecules in the sample 210. In various instances, the opto-fluidic instrument or system 200 can be a system configured to facilitate the experimental conditions conducive for the detection of the target molecules. For example, the opto-fluidic instrument or system 200 can include a fluidics module 230, an optics module 240, a sample module 250, and an ancillary module 260, and these modules may be operated by a system controller 220 to create the experimental conditions for hybridization probe-based detection and/or base-by-base sequencing of nucleic acid molecules in the sample 210, as well as to facilitate the imaging of the sample (e.g., by an imaging system of the optics module 240). In various instances, the various modules of the opto-fluidic instrument or system 200 may be separate components in communication with each other, or at least some of them may be integrated together.

In various instances, the sample module 250 may be configured to receive the sample 210 into the opto-fluidic instrument or system 200. For instance, the sample module 260 may include a sample interface module (SIM) that is configured to receive a sample device (e.g., cassette) onto which the sample 210 can be deposited. That is, the sample 210 may be placed in the opto-fluidic instrument or system 200 by depositing the sample 210 (e.g., the sectioned tissue) on a sample device that is then inserted into the SIM of the sample module 250. In some instances, the sample module 250 may also include an X-Y stage onto which the SIM is mounted. The X-Y stage may be configured to move the SIM mounted thereon (e.g., and as such the sample device containing the sample 210 inserted therein) in perpendicular directions along the two-dimensional (2D) plane of the opto-fluidic instrument or system 200.

The experimental conditions that are conducive for the detection of the molecules in the sample 210 may depend on the target molecule detection technique that is employed by the opto-fluidic instrument or system 200. For example, in various instances, the opto-fluidic instrument or system 200 can be a system that is configured to detect molecules (e.g., by detecting hybridization probes that hybridize to nucleic molecules (e.g., barcode sequences) and/or by nucleotides incorporated into extending sequencing primers using an identifier sequence as a template) in the sample 210.

In various instances, the fluidics module 230 may include one or more components that may be used for storing the reagents, as well as for transporting said reagents to and from the sample device containing the sample 210. For example, the fluidics module 230 may include reservoirs configured to store the reagents, as well as a waste container configured for collecting the reagents (e.g., and other waste) after use by the opto-fluidic instrument or system 200 to analyze and detect the molecules of the sample 210. Further, the fluidics module 230 may also include pumps, tubes, pipettes, etc., that are configured to facilitate the transport of the reagent to the sample device (e.g., and as such the sample 210). For instance, the fluidics module 230 may include pumps (“reagent pumps”) that are configured to pump washing/stripping reagents to the sample device for use in washing/stripping the sample 210 (e.g., as well as other washing functions such as washing an objective lens of the imaging system of the optics module 240).

In various instances, the ancillary module 260 can be a cooling system of the opto-fluidic instrument or system 200, and the cooling system may include a network of coolant-carrying tubes that are configured to transport coolants to various modules of the opto-fluidic instrument or system 200 for regulating the temperatures thereof. In such cases, the fluidics module 230 may include coolant reservoirs for storing the coolants and pumps (e.g., “coolant pumps”) for generating a pressure differential, thereby forcing the coolants to flow from the reservoirs to the various modules of the opto-fluidic instrument or system 200 via the coolant-carrying tubes. In some instances, the fluidics module 230 may include returning coolant reservoirs that may be configured to receive and store returning coolants, e.g., heated coolants flowing back into the returning coolant reservoirs after absorbing heat discharged by the various modules of the opto-fluidic instrument or system 200. In such cases, the fluidics module 230 may also include cooling fans that are configured to force air (e.g., cool and/or ambient air) into the returning coolant reservoirs to cool the heated coolants stored therein. In some instance, the fluidics module 230 may also include cooling fans that are configured to force air directly into a component of the opto-fluidic instrument or system 200 so as to cool said component. For example, the fluidics module 230 may include cooling fans that are configured to direct cool or ambient air into the system controller 220 to cool the same.

As discussed above, the opto-fluidic instrument or system 200 may include an optics module 240 which include the various optical components of the opto-fluidic instrument or system 200, such as but not limited to a camera, an illumination module (e.g., LEDs), an objective lens, and/or the like. The optics module 240 may include a fluorescence imaging system that is configured to image the fluorescence emitted by the detectably labeled nucleotides are incorporated in extending sequencing primers in the sample 210 after the detectable labels are excited by light from the illumination module of the optics module 240.

In some instances, the optics module 240 may also include an optical frame onto which the camera, the illumination module, and/or the X-Y stage of the sample module 250 may be mounted.

In various instances, the system controller 220 may be configured to control the operations of the opto-fluidic instrument or system 200 (e.g., and the operations of one or more modules thereof). In some instances, the system controller 220 may take various forms, including a processor, a single computer (or computer system), or multiple computers in communication with each other. In various instances, the system controller 220 may be communicatively coupled with data storage, set of input devices, display system, or a combination thereof. In some cases, some or all of these components may be considered to be part of or otherwise integrated with the system controller 220, may be separate components in communication with each other, or may be integrated together. In other examples, the system controller 220 can be, or may be in communication with, a cloud computing platform.

In various instances, the opto-fluidic instrument or system 200 may analyze the sample 210 and may generate the output 270 that includes indications of the presence of the target molecules in the sample 210. For instance, with respect to instances discussed above where the opto-fluidic instrument or system 200 employs a sequencing technique for detecting molecules, the opto-fluidic instrument or system 200 may cause the sample 210 to undergo successive sequencing cycles, where during the same sequencing cycle the sample is imaged to detect signals associated with nucleotide binding and/or incorporation events at some locations in the sample 210, as well as to detect an absence of signals at other locations in the sample. In such cases, the output 270 may include a series of optical signals (e.g., a code word) specific to each identifier sequence (e.g., a barcode sequence), which allow the identification of the target molecules.

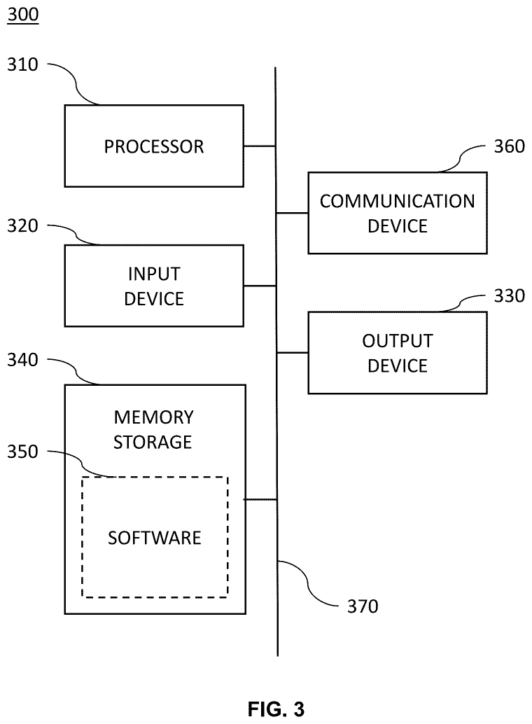

FIG. 3 illustrates an example of a computing device or system in accordance with one or more examples of the disclosure. Device 300 can be a host computer connected to a network. Device 300 can be a client computer or a server. As shown in FIG. 3, device 300 can be any suitable type of microprocessor-based device, such as a personal computer, workstation, server, or handheld computing device (portable electronic device), such as a phone or tablet. The device can include, for example, one or more of processor 310, input device 320, output device 330, memory/storage 340, and communication device 360. Input device 320 and output device 330 can generally correspond to those described above, and they can either be connectable or integrated with the computer.

Input device 320 can be any suitable device that provides input, such as a touch screen, keyboard or keypad, mouse, or voice-recognition device. Output device 330 can be any suitable device that provides output, such as a touch screen, haptics device, or speaker.

Storage 340 can be any suitable device that provides storage, such as an electrical, magnetic, or optical memory including a RAM, cache, hard drive, or removable storage disk. Communication device 360 can include any suitable device capable of transmitting and receiving signals over a network, such as a network interface chip or device. The components of the computer can be connected in any suitable manner, such as via a physical bus 370 or wirelessly.

Software 350, which can be stored in memory/storage 340 and executed by processor 310, can include, for example, the programming that embodies the functionality of the present disclosure (e.g., as embodied in the methods and systems described above). Software 350 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 340, that can contain or store programming for use by or in connection with an instruction execution system, apparatus, or device.

Software 350 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 300 can implement any operating system suitable for operating on the network. Software 350 can be written in any suitable programming language, such as C, C++, Java, or Python. In various implementations, application software embodying the functionality of the present disclosure can be deployed in different configurations, such as in a client/server arrangement or through a web browser as a web-based application or web service, for example.

### IX. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value typical of experiments or measurements made in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Detecting Errors and/or Noise During In Situ Nucleic Acid Sequencing

This example provides a workflow for real-time monitoring necessary for sequencing by synthesis in-situ (SBSIS). The provided workflow (e.g., incomplete functional group cleavage) may provide certain advantages such as real-time knowledge of nucleic acid sequencing, allowing for adjustment of a sequencing algorithm to account for errors and/or noise.

A tissue sample is obtained and cryosectioned onto a glass slide for processing. The tissue is fixed by incubating in 3.7% paraformaldehyde (PFA). To prepare for probe hybridization, a wash buffer is added to the tissue sample. The washed tissue sample is then contacted with circularizable probes (e.g., circularizable padlock probes) targeting either a gene of interest or, in the case of the control probe, a housekeeping gene (e.g., actB) (FIG. 1A and FIG. 1B). The barcode sequence identifies the target analyte (e.g., actB) within the tissue sample. The circularizable probes are allowed to hybridize to the target analyte (e.g., actB). The tissue sample is then contacted with a ligation reaction mix including ligase, and the circularizable probes are ligated to form circular templates for rolling circle amplification (RCA). The tissue sample is then incubated with an RCA mixture containing a Phi29 DNA polymerase and dNTP for RCA of the circularized probes. From this amplification, the RCA product (e.g., RCP) comprises the template nucleic acid molecule and a barcode sequence.

For in situ sequencing of the control probe, the tissue sample is washed and then contacted with a first sequencing primer comprising a first fluorescent signal (e.g., 5′ near-ultraviolet (NUV) fluorophore) detectable in a first channel, a repeating sequence of nucleotides and a modified 3′ reversibly terminated nucleotide, wherein the nucleotide comprises, e.g., a dibenzocyclooctynyl (DBCO) group attached to the base of the nucleotide via a disulfide linker. For the purines, the DBCO modification is attached to the N7 position of the base indirectly via a disulfide linker, and for pyrimidines the modification is attached to the C5 position of the base indirectly via a disulfide linker. The primer is allowed to hybridize to the template nucleic acid molecule in the RCP. Thus, the NUV fluorophore allows for rapid spatial identification from other RCPs bound by sequencing primers.

The tissue sample is washed and then contacted (in one or more steps) with a polymerase and a first plurality of modified nucleotides that each include an azido group. The plurality of modified nucleotides includes a corresponding detection channel for each nucleotide base: three different fluorophores and one “dark” label (e.g., A (second channel), T (third channel), C (fourth channel), G (dark)) (FIG. 2). A modified nucleotide molecule of the plurality and the polymerase are allowed to form a complex with the primer hybridized to the template nucleic acid molecule. The tissue sample is then washed to remove polymerase and unbound modified nucleotide molecules of the first plurality.

Fluorescence imaging is used to detect a signal associated with the presence of the modified nucleotide molecule bound to the primer. Images for each of a plurality of detection channels configured to detect signals arising from labels (e.g., fluorescent dyes) conjugated to nucleotide molecules present in the complex are acquired in each cycle of a multicycle sequencing reaction.

The control sequence comprises a known sequence comprising a repeating sequence of nucleotides four different nucleobases (e.g., a repeating sequence of ATCG). An expected signal for the first nucleobase type comprises a greater signal intensity in the second channel than the third and fourth channels, an expected signal for the second nucleobase type is a greater signal intensity in the third channel than the second channel and fourth channels, an expected signal for the third nucleobase type is a greater signal in the fourth channel than the second and third channels, and an expected signal intensity for the fourth nucleobase type is a minimal signal in the second, third, and fourth channels

Thus, the instrument will know a priori the exact signal that should be observed for each RCP observed in an NUV channel. While the exact sequence of the repeating sequence of nucleotides is irrelevant, representation of all nucleotides allows for the instrument to detect a control signal for each nucleotide. Any variation of the expected signal will inform the instrument as to the expected degree of chromatic blur and phasing to be expected in other circularizable probe (e.g., padlock probe) sequencing reactions.

For example, in a situation where there is perfect efficiency of nucleotide incorporation and complete cleavage over many cycles, the expected signal ratio would be as shown in Table 1. In this example, the known sequence that is sequenced using the control sequencing primer is a repeating sequence of “ATCG,” wherein A is labeled with a fluorophore that is detected at wavelength 590 nm, T is labeled with a fluorophore that is detected at wavelength 532 nm, C is labeled with a fluorophore that is detected at wavelength 647, and G not fluorescently labeled (dark)

| | |
|---|---|
| Expected signal ratio | Observed signal ratio |

| Cycle | 590 | 532 | 647 | 590 | 532 | 647 |
|---|---|---|---|---|---|---|
| 1 | 1.00 | 0 | 0 | 1.00 | 0 | 0 |
| 2 | 0 | 1.00 | 0 | 0 | 1.00 | 0 |
| 3 | 0 | 0 | 1.00 | 0 | 0 | 1.00 |
| 4 | 0 | 0 | 0 | 0 | 0 | 0 |
| 5 | 1.00 | 0 | 0 | 1.00 | 0 | 0 |
| 6 | 0 | 1.00 | 0 | 0 | 1.00 | 0 |
| 7 | 0 | 0 | 1.00 | 0 | 0 | 1.00 |
| 8 | 0 | 0 | 0 | 0 | 0 | 0 |
| 9 | 1.00 | 0 | 0 | 1.00 | 0 | 0 |
| 10 | 0 | 1.00 | 0 | 0 | 1.00 | 0 |
| 11 | 0 | 0 | 1.00 | 0 | 0 | 1.00 |
| 12 | 0 | 0 | 0 | 0 | 0 | 0 |

In another example, there may be minor errors in incorporation of nucleotides and/or cleavage of function groups. In this situation, the observed signal intensity may instead be as shown in Table 2.

| | |
|---|---|
| Expected signal ratio | Observed signal ratio |

| Cycle | 590 | 532 | 647 | 590 | 532 | 647 |
|---|---|---|---|---|---|---|
| 1 | 1.00 | 0 | 0 | 1.00 | 0 | 0 |
| 2 | 0 | 1.00 | 0 | 0.03 | 0.97 | 0 |
| 3 | 0 | 0 | 1.00 | 0.03 | 0.02 | 0.95 |
| 4 | 0 | 0 | 0 | 0.02 | 0.01 | 0.03 |
| 5 | 1.00 | 0 | 0 | 0.98 | 0 | 0.02 |
| 6 | 0 | 1.00 | 0 | 0.06 | 0.93 | 0.01 |
| 7 | 0 | 0 | 1.00 | 0.06 | 0.06 | 0.88 |
| 8 | 0 | 0 | 0 | 0.05 | 0.05 | 0.06 |
| 9 | 1.00 | 0 | 0 | 0.91 | 0.04 | 0.05 |
| 10 | 0 | 1.00 | 0 | 0.06 | 0.90 | 0.04 |
| 11 | 0 | 0 | 1.00 | 0.05 | 0.06 | 0.89 |
| 12 | 0 | 0 | 0 | 0.04 | 0.05 | 0.07 |

In this situation, the machine may recognize that the barcode will be incorrectly sequenced based on the accumulation of aberrant signal (phasing) passing a predetermined threshold value. For example, for the sequencing reaction shown in Table 2, the machine may recognize phasing is too great after cycle 6 (i.e., an observed signal ratio of <0.95 for the expected signal).

Thus, in a non-control sequence, the first nucleotide is assigned at the position if an intensity of at least a threshold value is measured in the second channel and intensities of below a cutoff value are measured in the third and the fourth channels, the second nucleotide is assigned at the position if an intensity of at least a threshold value is measured in the third channel and intensities of below a cutoff value are measured in the second and the fourth channels, the third nucleotide is assigned at the position if an intensity of at least a threshold value is measured in the fourth channel and intensities of below a cutoff value are measured in the first and the second channels, the fourth nucleotide is assigned at the position if an intensity of no more than a cutoff value is measured in all of the second, the third, and the fourth channels, and an unknown nucleotide (“N”) is assigned at the position if the criteria of none of the above criteria is met. These threshold values and cutoff values are determined based on the noise detected in the sequencing reactions for the control sequence.

As the repeating pattern (e.g., ATCGATCG . . . ) is repeatedly sequenced in the control sequence, the threshold values and cutoff values for base calling for non-control sequences can be adjusted based on the changing amount of noise in the sequencing reaction for the control sequence. For example, for base-calling of a non-control sequence in a reaction performed as part of the same sequencing reaction shown in Table 2, in a first cycle set, cycles 1-4, “C” may be called when a threshold observed signal of at least 0.95 is detected in the 647 channel (0.95 is the observed ratio for the known “C” in the first cycle set). Additionally, “G” may be called when no more than a cutoff observed ratio of 0.03 is observed in any of the three channels (as observed in the known “G” of the first cycle set). This algorithm may be adjusted as the pattern of sequencing the four bases is repeated. For example, in the second cycle set, cycles 5-8, “C” may be called when at least a threshold observed ratio of 0.88 is detected in the 647 channel (as observed for the known “C” of the second cycle set), and “G” may be called when no more than a cutoff observed ratio of 0.06 is observed in any of the three channels (as observed in the known “G” for the second cycle set). In this example of adjusted base calling algorithm, the threshold and cutoff values match exact observed values for the control. However, a padding can also be used, such as a threshold value for calling “C” that is 10% lower than an observed ratio during the “C” control of that cycle set (this would change the 647 channel threshold from 0.95 to 0.855 for calling a “C” during the first cycle set), and a cutoff value for calling “G” that is 10% higher than the observed ratio in any of the channels during the “G” control of that cycle set (this would change the cutoff for all three channels from 0.03 to 0.033 for calling a “G” in the first cycle set).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method, comprising:
a) contacting a biological sample, wherein the biological sample comprises at a first location a plurality of copies of a control sequence comprising a known sequence, with sequencing primers comprising:
(i) a control sequencing primer labeled with a first fluorescent dye, wherein the control sequencing primer binds adjacent to the control sequence; and
(ii) a second sequencing primer, wherein the second sequencing primer binds adjacent to a sequence of interest;
b) detecting the first fluorescent dye at the first location, thereby identifying the first location;
c) performing a plurality of cycles of a nucleic acid sequencing reaction by extending the sequencing primers; and
d) for a cycle of the plurality of cycles at the identified first location, comparing a signal from the nucleic acid sequencing reaction to an expected signal from the known sequence, thereby determining noise in the signal and/or identifying an error in the nucleic acid sequencing reaction.

2. The method of claim 1, wherein the plurality of cycles comprises at least four cycles.

3. The method of claim 1, wherein at least one position of the known sequence is adenine (A), at least one position of the known sequence is thymine (T) or uracil (U), at least one position of the known sequence is guanine (G), and at least one position of the known sequence is cytosine (C).

4. (canceled)

5. The method of claim 1, wherein the plurality of copies of the control sequence comprises locally amplified copies of the control sequence.

6. The method of claim 1, wherein the plurality of copies of the control sequence are in a rolling circle amplification product.

7. The method of claim 1, wherein the biological sample further comprises a plurality of copies of the sequence of interest.

8-9. (canceled)

10. The method of claim 1, wherein each cycle of the nucleic acid sequencing reaction comprises determining a fluorescent signal intensity indicative of incorporation or binding of a nucleotide of a particular nucleobase type at one or more locations comprising the first location.

11-18. (canceled)

19. The method of claim 1, wherein the first fluorescent dye is detectable in a first channel, and wherein the signal from the nucleic acid sequencing reaction comprises detecting a fluorescent signal intensity indicative of incorporation or binding of nucleotide of a particular nucleobase type in additional channels comprising a second channel, a third channel, and a fourth channel.

20. (canceled)

21. The method of claim 1, wherein d) comprises identifying an error in the nucleic acid sequencing reaction.

22. The method of claim 21, wherein the signal intensity at the first location is measured in: (i) a channel associated with a nucleobase type of a known nucleotide of the control sequence and (ii) one or more channels associated with a different nucleobase type from that of the known nucleotide, and the error is identified based on the signal intensity being greater in one of the one or more channels associated with a different nucleobase type than the channel associated with the nucleobase type of the known nucleotide.

23. The method of claim 1, wherein d) comprises identifying a phasic synchrony error in sequence data captured from the plurality of copies of the control sequence.

24-25. (canceled)

26. The method of claim 1, wherein d) comprises determining noise in the signal.

27. The method of claim 26, wherein the noise is determined based on a measured signal intensity in a channel associated with a different nucleobase type than the nucleobase type of the known nucleotide of the known sequence.

28. The method of claim 1, wherein the method further comprises:
e) generating at least one sequence read for the sequence of interest; and
f) correcting the sequence read by removing a basecall at a position from a cycle of the sequencing reaction having the error and/or noise identified in d).

29. The method of claim 28, wherein removing the basecall comprises assigning an “N” at the position in the position of the cycle having the error and/or noise identified in d).

30. The method of claim 1, wherein d) comprises assigning a quality score to the sequence data captured from the plurality of copies of the control sequence based on the comparison between the detected fluorescent signal intensities from the nucleic acid sequencing reaction at the first location to the control sequence.

31. (canceled)

32. The method of claim 1, wherein d) is performed after each of multiple cycles of the plurality of cycles of the nucleic acid sequencing reaction.

33. (canceled)

34. The method of claim 1, comprising performing cycle-by-cycle phasing corrections by determining the presence or absence of a phasing error in d) for each of multiple cycles of the nucleic acid sequencing reaction, and
for each of the multiple cycles where a phasing error was identified, calculating a new phasing correction based on the identified phasing error of the respective cycle; and
applying the new phasing correction to a subsequent cycle.

35-36. (canceled)

37. The method of claim 1, further comprising, prior to contacting the biological sample with sequencing primers in a), contacting the biological sample with a control probe or probe set that binds to a control nucleic acid molecule, wherein the control probe or probe set comprises a complement of the control sequence, and locally amplifying the control probe or probe set to generate a product of the control probe or probe set comprising the plurality of copies of the control sequence.

38-40. (canceled)

41. The method of claim 37, wherein the method comprises contacting the biological sample with the control probe or probe set and the additional probe or probe set at a ratio of between about 1:10 and 1:100,000.

42-45. (canceled)

46. The method of claim 1, wherein the first fluorescent dye is an ultraviolet (UV), deep ultraviolet (DUV) or near ultraviolet (NUV) dye.

47-49. (canceled)

50. The method of claim 1, wherein the biological sample comprises a layer of cells deposited on a surface.

51-64. (canceled)


SEQUENCE LISTING (6 sequences)

SEQ ID NO: 1 - protein, 306 aa, Thermus aquaticus
GMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLXAFLERLEFGSLLHEFGLLESPKXLXEAPWPPPERAFVP

SEQ ID NO: 2 - protein, 575 aa, unidentified [sequence omitted]

SEQ ID NO: 3 - protein, 775 aa, Thermococcus sp.
  source 1..775: note = 9N-7
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 4 - protein, 928 aa, unidentified [sequence omitted]

SEQ ID NO: 5 - protein, 560 aa, unidentified
AFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLGRLEDLKSKTLGDLVREAEKIFNKRETPEEREERIRRETEEKEERRRTVDEQKEKERDRRRHREMSKLLATVVIGQEQDRQEGERKRPQLDKDQCAYCKEKGHWAKDCPKKPRGPRGPRPQTSLLTLGDXGGQGQDPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVSLGFTWLSDFPQAWAESGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFR

SEQ ID NO: 6 - protein, 259 aa, Human immunodeficiency virus 1
PISPIEPVPVKLKPGMDGPKVKQWPLTEEKIKALVEICTEMEKEGKISKIGPENPYNTPVFAIKKKDSTRWRKLVDFRELNKRTQDFWEVQLGIPHPAGLKKKRSVTVLDVGDAYFSVPLDKEFRKYTAFTIPSINNETPGIRYQYNVLPQGWKGSPAIFQSSMTKILEPFRKQNPDIVIYQYMDDLYVGSDLEIGQHRTKIEELRQHLLKWGFTTPDKKHQKEPPFLWMGYEHHPDKWTVQPIVLPEKDSWTVNDIQK